(19) United States
(12) Patent Application Publication
(10) Pub. No.: US 2004/0018622 A1
(43) Pub. Date: Jan. 29, 2004

(54) Spliceosome mediated RNA trans-splicing for correction of skin disorders

(76) Inventors: Lloyd G. Mitchell, Bethesda, MD (US); Madaiah Puttaraju, Germantown, MD (US); Guenter Dallinger, Linz (AT); Alfred Klausegger, Salzburg (AT); Johann Bauer, Salzburg (AT)

Correspondence Address: BAKER & BOTTS, 30 ROCKEFELLER PLAZA, NEW YORK, NY, 10112

(21) Appl. No.: 10/198,447

(22) Filed: Jul. 17, 2002

(51) Int. Cl. A61K 48/00; C12N 5/08

(52) U.S. Cl. 435/371; 514/44; 424/93.21

(57) ABSTRACT

The present invention provides methods and compositions for generating novel nucleic acid molecules through targeted spliceosomal mediated RNA trans-splicing. The compositions of the invention include pre-trans-splicing molecules (PTMs) designed to interact with a target precursor messenger RNA molecule (target pre-mRNA) and mediate a trans-splicing reaction resulting in the generation of a novel chimeric RNA molecule (chimeric RNA). In particular, the PTMs of the present invention are genetically engineered to interact with a specific target pre-mRNA expressed in cells of the skin so as to result in correction of genetic defects responsible for a variety of different skin disorders. The compositions of the invention further include recombinant vectors systems capable of expressing the PTMs of the invention and cells expressing said PTMs. The methods of the invention encompass contacting the PTMs of the invention with specific target pre-mRNA expressed within cells of the skin under conditions in which a portion of the PTM is trans-spliced to a portion of the target pre-mRNA to form a chimeric RNA molecule wherein the genetic defect in the specific gene has been corrected. The present invention is based on the successful trans-splicing of the collagen XVII pre-mRNA thereby establishing the usefulness of trans-splicing for correction of skin specific genetic defects. The methods and compositions of the present invention can be used in gene therapy for treatment of specific disorders of the skin, i.e., genodermatoses, such as epidermal fragility disorders, keratinization disorders, hair disorders and pigmentation disorders as well as cancers of the skin.

### 1. INTRODUCTION

The present invention provides methods and compositions for generating novel nucleic acid molecules through targeted spliceosomal mediated RNA trans-splicing. The compositions of the invention include pre-trans-splicing molecules (PTMs) designed to interact with a target precursor messenger RNA molecule (target pre-mRNA) and mediate a trans-splicing reaction resulting in the generation of a novel chimeric RNA molecule (chimeric RNA). In particular, the PTMs of the present invention are genetically engineered to interact with a specific target pre-mRNA expressed in cells of the skin so as to result in correction of genetic defects responsible for a variety of different skin disorders. The compositions of the invention further include recombinant vectors systems capable of expressing the PTMs of the invention and cells expressing said PTMs. The methods of the invention encompass contacting the PTMs of the invention with specific target pre-mRNA expressed within cells of the skin under conditions in which a portion of the PTM is trans-spliced to a portion of the target pre-mRNA to form a chimeric RNA molecule wherein the genetic defect in the specific gene has been corrected. The present invention is based on the successful trans-splicing of the collagen XVII pre-mRNA thereby establishing the usefulness of trans-splicing for correction of skin specific genetic defects. The methods and compositions of the present invention can be used in gene therapy for treatment of specific disorders of the skin, i.e., genodermatoses, such as epidermal fragility disorders, keratinization disorders, hair disorders and pigmentation disorders as well as proliferative disorders of the skin such as cancer and psoriasis of the skin.

### 2. BACKGROUND OF THE INVENTION

Significant progress has recently been made towards better understanding the genetic basis of heritable skin disorders. An understanding of the underlying mutations responsible for specific skin disorders has provided the basis for cutaneous gene therapy. Because of the easy accessibility of skin and the fact that skin cells, such as keratinocytes and dermal fibroblasts, can be easily grown in culture, the skin provides an ideal tissue for gene therapy.

Epidermolysis bullosa (EB) is the term applied to a heterogeneous group of inherited skin disorders in which minor trauma leads to blistering of skin and mucous membranes. Depending on the level of tissue cleavage, EB can be divided into three main groups: (i) EB simplex with blister formation occurring in the basal keratinocyte, (ii) junctional EB (JEB) with blister formation in the lamina lucida and (iii) EB dystrophicans with blister formation below the lamina densa.

JEB patients are divided into two main groups, Herlitz JEB and generalized atrophic benign EB (GABEB). Patients diagnosed with the former disease usually die within their first year of life, whereas the latter diagnosis is associated with a better prognosis and a tendency for improvement during life. Initial observations describing reduced expression of bullous pemphigoid antigen 2 (BPAG2), identified as type XVII collagen, in patients suffering from GABEB were followed by the identification of mutations in the gene coding for BPAG2 (Col17A1). To date, a number of different mutations in the Col17A1 have been identified leading to the establishment of a mutation database, which has facilitated the analysis of the effects of specific mutations on the clinical presentation of nH-JEB. For example, it has been determined that stop codon mutations or mutations leading to downstream stop codons on both alleles are associated with the original “GABEB” phenotype.

In addition, EB simplex with late onset muscular dystrophy (EBS-MD) patients have been characterized with mutations in the plectin gene. Some of these patients feature compound heterozygosity for a three base-pair insertion at position 1287 (1287ins3) leading to the insertion of leucine as well as missense mutation, Q1518X causing the insertion of a stop codon in the plectin coding region (Bauer, J W et al., 2001 Am J Pathol 158: 617-625).

In skin gene therapy, most efforts to date have attempted to deliver full length cDNA copies of the affected gene using retroviral vectors. However, the delivery of full length cDNA in skin therapy is often limited by the size of the mRNA (or cDNA), for example, the plectin mRNA is 14.8 kb, the type VII collagen mRNA is 9.2 kb and the type XVII collagen mRNA is 6.5 kb. The size of these genes, mutated in patients with various forms of EB, and their regulatory elements are beyond the capacity of delivery systems suitable for skin gene therapy using retroviral or adeno-associated viral vectors. Therefore, it would be advantageous to reduce the size of the therapeutic sequence that has to be delivered.

It is also critical that the genes implicated in cutaneous blistering disorders and targeted for gene therapy are only expressed by keratinocytes of a specific epidermal layer. For example, ectopic expression of such genes may lead to disordered epithelial polarity. One possible way to address the problem of keratinocyte specific expression is to use specific regulatory elements to direct transgene expression. However, the use of such promoters further increases the size of the insert in a therapeutic vector.

For the Col17A1 gene, alternative approaches to gene correction have been described. Notably, there are natural mechanisms by which mutations have been corrected in the Col17A1 gene validating the concept of gene therapy. For example, Jonkman et al., (1997, Cell 88:543-551) reported on a patient who had patches of normal appearing skin in a symmetrical leaf-like pattern on the upper extremities. The underlying mutations in the Col17A1 gene had been identified as R1226X paternally, and 1706delA, maternally. In the clinical unaffected areas of the skin about 50% of the basal cells were expressing type XVII collagen at a reduced level due to a mitotic gene conversion surrounding the maternal mutation, thus leading to loss of heterozygosity in this area. These observations suggest that expression of less than 50% of full length type XVII collagen is sufficient to correct the phenotypic expression of nH-JEB. In addition, a partly successful gene correction by the keratinocyte splicing machinery has been described in patients with the homozygous R785X mutation in the Col17A1 gene (Ruzzi L et al., 2001 J. Invest Dermatol 116:182-187). In these patients, the exclusion of exon 33, harboring the mutation, leads to an unusual mild phenotype, although there is only 3-4% of detectable type XVII collagen protein. Similar in frame skipping of exons has also been reported for patients with mutations in the Col17A1 and LAMB3 gene.

Until recently, the practical application of targeted trans-splicing to modify specific target genes was limited to group I ribozyme-based mechanisms. Using the Tetrahymena group I ribozyme, targeted trans-splicing was demonstrated in E. coli. (Sullenger B. A. and Cech. T. R., 1994, Nature 341:619-622), in mouse fibroblasts (Jones, J. T. et al., 1996, Nature Medicine 2:643-648), human fibroblasts (Phylactou, L. A. et al., 1998 Nature Genetics 18:378-381) and human erythroid precursors (Lan et al., 1998, Science 280:1593-1596). While many applications of targeted RNA trans-splicing driven by modified group I ribozymes have been explored, targeted trans-splicing mediated by native mammalian splicing machinery, i.e., spliceosomes, is now being actively developed.

Spliceosomal mediated trans-splicing utilizes the endogenous cellular splicing machinery to repair inherited genetic defects at the RNA level by replacing mutant exon or exons. The use of such techniques has a number of advantages over the conventional gene therapy approaches. For example, the repaired product is always under endogenous regulation and correction will only occur in cells endogenously expressing the target pre-mRNA. In addition, genetic diseases can be corrected regardless of the mode of inheritance. Finally, the use of trans-splicing reduces the size of the transgene into an expression vector.

U.S. Pat. Nos. 6,083,702, 6,013,487 and 6,280,978 describe the use of PTMs to mediate a trans-splicing reaction by contacting a target precursor mRNA to generate novel chimeric RNAs. The present invention provides specific PTM molecules designed to correct specific defective genes expressed within cells of the skin and associated with skin disorders. The specific PTMs of the invention may be used to treat a variety of different skin disorders such as genodermatoses including but not limited to epidermal fragility disorders, keratinization disorders, hair disorders, pigmentation disorders and cancer disorders.

### 3. SUMMARY OF THE INVENTION

The present invention relates to compositions and methods for generating novel nucleic acid molecules through spliceosome-mediated targeted trans-splicing. In particular, the compositions of the invention include pre-trans-splicing molecules (hereinafter referred to as “PTMs”) designed to interact with a specific target pre-mRNA molecule expressed within cells of the skin (hereinafter referred to as “skin cell specific pre-mRNA”) and mediate a spliceosomal trans-splicing reaction resulting in the generation of a novel chimeric RNA molecule (hereinafter referred to as “chimeric RNA”). Skin specific pre-mRNA molecules include, but are not limited to, those transcribed from the collagen genes, i.e., type VII collagen, type XVII collagen (Col17A1), laminin and plectin genes to name a few. The invention is based on the successful targeted trans-splicing of the endogenous Col17A1 pre-mRNA in keratinocytes of the skin, however, the methods and compositions of the invention may also be used to target defective genes in other types of skin cells, i.e., fibroblasts, melanocytes, dermal papilla cells, nerve cells and blood cells.

The compositions of the invention include PTMs designed to interact with a skin specific target pre-mRNA molecule and mediate a spliceosomal trans-splicing reaction resulting in the generation of a novel chimeric RNA molecule. Such PTMs are designed to correct genetic defects in a skin specific gene. The general design, construction and genetic engineering of PTMs and demonstration of their ability to successfully mediate trans-splicing reactions within the cell are described in detail in U.S. Pat. Nos. 6,083,702, 6,013,487 and 6,280,978 as well as patent Ser. Nos. 09/756,095, 09/756,096, 09/756,097 and 09/941,492, the disclosures of which are incorporated by reference in their entirety herein.

The methods of the invention encompass contacting the PTMs of the invention with a skin cell specific target pre-mRNA under conditions in which a portion of the PTM is trans-spliced to the target pre-mRNA to form a novel chimeric RNA. The methods of the invention comprise contacting the PTMs of the invention within a cell expressing a skin cell specific target pre-mRNA under conditions in which the PTM is taken up by the cell and a portion of the PTM is trans-spliced to a portion of the target pre-mRNA to form a novel chimeric RNA molecule that results in correction of a skin cell specific genetic defect. Alternatively, nucleic acid molecules encoding PTMs may be delivered into a target cell followed by expression of the nucleic acid molecule to form a PTM capable of mediating a trans-splicing reaction. The PTMs of the invention are genetically engineered so that the novel chimeric RNA resulting from the trans-splicing reaction encodes a protein that complements a defective or inactive skin cell specific protein within the cell. The methods and compositions of the invention can be used in gene repair for the treatment of various skin disorders, such as epidermolysis bullosa.

### 4. BRIEF DESCRIPTION OF THE DRAWINGS

FIG. 1. Schematic representation of different trans-splicing reactions. (a) trans-splicing reactions between the target 5′ splice site and PTM's 3′ splice site, (b) trans-splicing reactions between the target 3′ splice site and PTM's 5′ splice site and (c) replacement of an internal exon by a double trans-splicing reaction in which the PTM carries both 3′ and 5′ splice sites. BD, binding domain; BP, branch point sequence; PPT, polypyrimidine tract; and ss, splice sites.

FIGS. 2A-D. Schematic representation of Col17A1 model constructs. Detailed structure of (FIG. 2A) the COLI7A1-lacZ target (lacZ-T1) for the β-gal model-system and of (FIG. 2B) the Col17A1 mini-gene target (T2). The relative position of primers lac9F, KI-3R and KI-5R are indicated. (FIG. 2C) Schematic diagram of a PTM for the β-gal test-system. (1; 2; 3) Detailed structures and sequences of the PTM1, 3 and 5 binding domains, respectively. (FIG. 2D) Schematic diagram of PTMs used in the Col17A1 mini-gene system (1; 2; 3). Detailed structures and sequences of the PTM2, 4 and 6 binding domains, respectively. Abbreviations: BP; branch point, PPT: polypyrimidine tract, ss: 5′ and 3′ splices sites, BD: binding domain.

FIGS. 3A-C. The β-gal test-system shows accurate trans-splicing at the RNA level and restoration of β-gal protein function in 293T cells using Col17A1 intron 51 as a target. FIG. 3A. Demonstration of cis- and trans-splicing in 293T cells using the β-gal test-system. One representative experiment of 5 experiments is shown. 30 ng and 300 ng of total RNA were used for the detection of cis- (left panel) or trans-splicing (right panel), respectively. Lane 1: Transfection experiment with vector alone. Lane 2: Transfection of LacZ-T1 alone. Lanes 3, 4, 5: Transfection of PTM1, 3 and 5 alone. Lane 6, 7, 8: Co-transfection of 2 μg LacZ-T1 and 2 μg of either PTM1, 3 or 5. Lane M: 100 bp DNA size marker. FIG. 3B. Upper panel: DNA sequence of cis-spliced lacZ-T1 target mRNA showing the correct splicing between the 5′ and 3′ exon and two in frame stop codons (underlined). The splice junction is indicated by an arrow. Lower panel: DNA sequence of trans-spliced mRNA showing the accurate trans-splicing and replacement of the stop codons. FIG. 3C. Restoration of β-gal activity is increased with respect to the length of the binding domain. β-gal activity representing the average of four independent transfection experiments. Lysates from 293T cells transfected with 2 μg of LacZ-T1, PTM3 and PTM5 alone, respectively or co-transfected with 2 μg target (LacZ-T1) and 2 μg of PTM; LacZ-T1+PTM1: 95.73 U/mg (+SD 30 U/mg) protein; LacZ-T1+PTM3: 117.52 U/mg (+SD 30 U/mg) protein. LacZ-T1+PTM5: 328.94 U/mg (+SD 50 U/mg) protein. (SD=standard deviation).

FIG. 4. Efficient and accurate trans-splicing between LacZ-T1 pre-mRNA and PTM5 RNA produces functional β-gal in epithelial 293T cells, in human keratinocytes and a GABEB cell-line in vitro. 293T cells (uppermost panel), human primary keratinocytes (middle panel) and a GABEB cell-line (lowest panel) were transfected with pcDNA3.1 vector (control), or co-transfected with LacZ-T1+PTM5. 25% of transfected 293T cells showed restoration of β-gal expression; while β-gal activity was restored in 5% of primary keratinocytes and the GABEB keratinocyte cell line. No β-gal activity was detected in the control cells.

FIGS. 5A-B. Trans-splicing between the T2 mini-gene pre-mRNA and pCol17-PTM's containing the cDNA sequence spanning exons 52 to 56 in 293T cells. FIG. 5A. Upper panel; Lane 1: Mock transfection with pcDNA3.1 vector. Transfection of either T2 or PTM2, PTM4 and PTM6 alone, showing correct cis-splicing of the target pre-mRNA in Lane 2 and the absence of cis-splicing products for all PTM's when transfected alone (lanes 3, 4 and 5), respectively. Lanes 6, 7 and 8 are showing co-transfection experiments of T2 and PTM2, PTM4, and PTM6 producing a fragment of the predicted length (568 bp) Lane M; 100 bp DNA size marker. Lower panel: Lane 1: Mock transfection experiment with pcDNA3.1 vector. RT-PCR fragments of trans-spliced product (574 bp) can be obtained from RNA prepared from co-transfection experiments using T2 as a target and either PTM2, PTM4, or PTM6 (Lanes 6, 7 and 8), respectively. Transfections of either T2 or PTM2, PTM4, and PTM6 alone showed no trans-splicing (Lanes 2, 3, 4 and 5). Lane M: 100 bp DNA size marker. FIG. 5B. Schematic drawing showing the binding sites of primers used for RT-PCR analysis of mini-gene cis- and trans-splicing.

FIG. 6A. Accurate trans-splicing restores β-gal activity in human keratinocytes. FIGS. 6A-B. Primary keratinocytes (I) β-gal activity in units/mg protein in human keratinocytes Lane 1: transfection of pcDNA3.1 vector alone. Lane 2: LacZ-T1 alone. Lane 3: PTM5 alone. Lane 4: Co-transfection of LacZ-T1 and PTM5 revealing a β-gal activity of 190 U/mg protein (+SD 50 U/mg). (II) RT-PCR analysis of total RNA prepared from the same experiment for cis-splicing (left panel) and trans-splicing (right panel). Control transfections included vector alone (Lane 1); LacZ-T1 alone (Lane 2) and PTM5 alone (Lane 3). Lane 4 shows a RT-PCR product of 298 nt length as predicted for accurate trans-splicing between the target and PTM5 (right picture). A 302 nt RT-PCR product is generated in Lane 2 (LacZ-T1 alone) and Lane 4 (LacZ-T1+PTM5) showing cis-splicing of the LacZ-T1 target (left picture). FIG. 6B. Immortalized GABEB keratinocytes (I) β-gal activity in units/mg protein in the GABEB cell-line. Lane 1: Transfection of pcDNA3.1 vector alone. Lane 2: LacZ-T1 alone. Lane 3: PTM5 alone. Lane 4; Co-transfection of LacZ-T1 and PTM5 revealing β-gal activity of 295.6 U/mg protein (+SD 60 U/mg). (II) RT-PCR analysis of total RNA prepared from the same experiment for cis-splicing (left panel) and trans-splicing (right panel). Control transfections included vector alone (Lane 1); LacZ-T1 alone (Lane 2) and PTM5 alone (Lane 4). Lane 3 showing the RT-PCR product of 298 nt length as predicted for accurate trans-splicing of target and PTM5 (right picture). RT-PCR for cis-splicing of LacZ-T1 shows a 302 nt product in lanes 2 (LacZ-T1 alone) and Lane 3 (LacZ-T1+PTM5) (left picture).

FIG. 7. Detection strategy for endogenous trans-splicing of the Col17A1 pre-mRNA in HaCatKC cells. Therapeutic molecule (PTM5) consists of Col7A1 binding domain 51, spacer element, branch point (BP) and polypyrimidine tract (PPT) followed by a functional part of β-galactosidase lacZ 3′ exon cloned into pcDNA3.1(−). This construct was transfected into HaCat cells. Pre-mRNA resulted in correct endogenously trans-spliced product of a genomic fragment spanning exon 1-51 and LacZ 3′ exon confirmed by semi-nested RT-PCR with primer 51-1F, lac6R and lac4R.

FIG. 8. Endogenous trans-splicing of Col17A1 pre-mRNA with PTM5. Sequence of correct endogenously trans-spliced product showing the splice junction between exon 51 with lacZ 3′ exon (A) and confirmation by restriction digestion of 226 bp RT-PCR product with Msel resulting in two fragments of 168 bp and 58 bp (B).

FIG. 9. Schematic of 5′ trans-splicing LacZ repair model for hereditary diseases.

FIG. 10. Target LacZ-T3 containing intron 9 of the plectin gene and lacZ-T4 used for optimizing trans-splicing and transfection conditions.

FIG. 11. LacZ-PTM3 (intron 9 specific binding domain) and lacZ-PTM4 (non-specific binding domain) for establishing optimal trans-splicing conditions.

FIG. 12. PLEC-PTM-5 for the introduction of the 1287ins3 mutation in 293T cells.

FIG. 13. PLEC-PTM-6 for repair of the 1287ins3 mutation in plectin deficient patient cells.

### 5. DETAILED DESCRIPTION OF THE INVENTION

The present invention relates to compositions comprising pre-trans-splicing molecules (PTMs) and the use of such molecules for generating novel nucleic acid molecules. The PTMs of the invention comprise (i) one or more target binding domains that are designed to specifically bind to a skin cell specific target pre-mRNA and (ii) a 3′ splice region that includes a branch point and a 3′ splice acceptor site and/or a 5′ splice donor site. The 3′ splice region may further comprise a polypyrimidine tract. In addition, the PTMs of the invention can be engineered to contain any nucleotide sequences such as those encoding a translatable protein product and one or more spacer regions that separate the RNA splice site from the target binding domain.

The methods of the invention encompass contacting the PTMs of the invention with a skin cell specific target pre-mRNA under conditions in which a portion of the PTM is trans-spliced to a portion of the target pre-mRNA to form a novel chimeric RNA that results in correction of a skin cell specific genetic defect. Such skin specific target pre-mRNA molecules include but are not limited to those encoding plectin, type XVII collagen, type VII collagen and laminin, to name a few.

### 5.1 Structure of the Pre-Trans-Splicing Molecules

The present invention provides compositions for use in generating novel chimeric nucleic acid molecules through targeted trans-splicing. The PTMs of the invention comprise (i) one or more target binding domains that targets binding of the PTM to a skin cell specific target pre-mRNA and (ii) a 3′ splice region that includes a branch point and a 3′ splice acceptor site and/or 5′ splice donor site. The 3′ splice region may additionally contain a polypyrimidine tract. The PTMs may also contain (a) one or more spacer regions that separate the splice site from the target binding domain, (b) mini-intron sequences, (c) ISAR (intronic splicing activator and repressor) consensus binding sites, and/or (d) ribozyme sequences. Additionally, the PTMs of the invention contain skin cell specific exon sequences designed to correct a skin cell specific genetic defect.

The present invention further provides methods and compositions for real time imaging of gene expression in cells of the skin. The compositions of the invention include pre-trans-splicing molecules designed to interact with a target precursor messenger RNA molecule expressed within a cell of the skin and mediate a trans-splicing reaction resulting in the generation of a novel chimeric RNA molecule designed to encode a reporter molecule. The PTMs of the invention are engineered to interact with target pre-mRNAs where the expression of the target pre-mRNA is correlated with a disease of the skin. Thus, the present invention provides methods and compositions for the diagnosis and/or prognosis of skin disease in a subject. Such skin diseases include, but are not limited to disorders resulting from aberrant gene expression, proliferative disorders such as cancers or psoriasis, or infectious diseases.

A variety of different PTM molecules may be synthesized for use in the production of a novel chimeric RNA which complements a defective or inactive skin cell specific protein. The general design, construction and genetic engineering of such PTMs and demonstration of their ability to mediate successful trans-splicing reactions within the cell are described in detail in U.S. Pat. Nos. 6,083,702, 6,013,487 and 6,280,978 as well as patent Ser. Nos. 09/941,492, 09/756,095, 09/756,096 and 09/756,097 the disclosures of which are incorporated by reference in their entirety herein.

As used herein, skin cell is defined as any of the different cell types found within the epidermal, dermal and/or first layer of the skin. Such skin cell types include, for example, melanocytes, keratinocytes, fibroblasts, blood vessel cells, hair follicle cells, neuronal cells of the skin and cancer cells of the skin.

The target binding domain of the PTM endows the PTM with a binding affinity. As used herein, a target binding domain is defined as any molecule, i.e., nucleotide, protein, chemical compound, etc., that confers specificity of binding and anchors the skin cell specific pre-mRNA target closely in space to the PTM so that the spliceosome processing machinery in the nucleus can trans-splice a portion of the PTM to a portion of the skin cell specific target pre-mRNA. The target binding domain of the PTM may contain multiple binding domains which are complementary to and in antisense orientation to the targeted region of the selected pre-mRNA. The target binding domains may comprise up to several thousand nucleotides. In preferred embodiments of the invention the binding domains may comprise at least 10 to 30 and up to several hundred or more nucleotides. The specificity of the PTM may be increased significantly by increasing the length of the target binding domain. For example, the target binding domain may comprise several hundred nucleotides or more. In addition, although the target binding domain may be “linear” it is understood that the RNA may fold to form secondary structures that may stabilize the complex thereby increasing the efficiency of splicing. A second target binding region may be placed at the 3′ end of the molecule and can be incorporated into the PTM of the invention. Absolute complementarily, although preferred, is not required. A sequence “complementary” to a portion of an RNA, as referred to herein, means a sequence having sufficient complementarity to be able to hybridize with the target pre-RNA, forming a stable duplex. The ability to hybridize will depend on both the degree of complementarity and the length of the nucleic acid (See, for example, Sambrook et al., 1989, Molecular Cloning, A Laboratory Manual, 2d Ed., Cold Spring Harbor Laboratory Press, Cold Spring Harbor, N.Y.). Generally, the longer the hybridizing nucleic acid, the more base mismatches with an RNA it may contain and still form a stable duplex. One skilled in the art can ascertain a tolerable degree of mismatch, length or structure of the duplex by use of standard procedures to determine the stability of the hybridized complex.

Binding may also be achieved through other mechanisms, for example, through triple helix formation, aptamer interactions, RNA lassos (see PCT application: PCT/US98/17268) antibody interactions or protein/nucleic acid interactions such as those in which the PTM is engineered to recognize a specific RNA binding protein, i.e., a protein bound to a specific target pre-mRNA. Alternatively, the PTMs of the invention may be designed to recognize secondary structures, such as for example, hairpin structures resulting from intramolecular base pairing between nucleotides within an RNA molecule.

In a specific embodiment of the invention, the target binding domain is complementary and in anti-sense orientation to sequences in close proximity to the region of the keratinocyte specific target pre-mRNA targeted for trans-splicing. In a specific embodiment of the invention, the target binding domain is complementary and in antisense orientation to keratinocyte specific target pre-mRNAs nucleotide sequences, including but not limited to plectin, type VII collagen, type XVII collagen (Col17A1), and laminin. For a review of skin disorders and known genetic defects see Uitto et al., (2000, Human Gene Therapy 11:2267-2275) the disclosure of which is incorporated by reference in its entirety herein.

The PTM molecule also contains a 3′ splice region that includes a branch point sequence and a 3′ splice acceptor AG site and/or a 5′ splice donor site. The 3′ splice region may further comprise a polypyrimidine tract. Consensus sequences for the 5′ splice donor site and the 3′ splice region used in RNA splicing are well known in the art (See, Moore, et al., 1993, The RNA World, Cold Spring Harbor Laboratory Press, p. 303-358). In addition, modified consensus sequences that maintain the ability to function as 5′ donor splice sites and 3′ splice regions may be used in the practice of the invention. Briefly, the 5′ splice site consensus sequence is AG/GURAGU (where A=adenosine, U=uracil, G=guanine, C=cytosine, R=purine and /=the splice site). The 3′ splice site consists of three separate sequence elements: the branch point or branch site, a polypyrimidine tract and the 3′ consensus sequence (YAG). The branch point consensus sequence in mammals is YNYURAC (Y=pyrimidine; N=any nucleotide). The underlined A is the site of branch formation. A polypyrimidine tract is located between the branch point and the splice site acceptor and is important for efficient branch point utilization and 3′ splice site recognition. Other pre-messenger RNA introns beginning with the dinucleotide AU and ending with the dinucleotide AC have been identified and referred to as U12 introns. U12 intron sequences as well as any sequences that function as splice acceptor/donor sequences may also be used to generate the PTMs of the invention.

A spacer region to separate the RNA splice site from the target binding domain may also be included in the PTM. The spacer region may be designed to include features such as stop codons which would block translation of an unspliced PTM and/or sequences that enhance trans-splicing to the target pre-mRNA.

In a preferred embodiment of the invention, a “safety” is also incorporated into the spacer, binding domain, or elsewhere in the PTM to prevent non-specific trans-splicing. This is a region of the PTM that covers elements of the 3′ and/or 5′ splice site of the PTM by relatively weak complementarity, preventing non-specific trans-splicing. The PTM is designed in such a way that upon hybridization of the binding/targeting portion(s) of the PTM, the 3′ and/or 5′ splice site is uncovered and becomes fully active.

The “safety” consists of one or more complementary stretches of cis-sequence (or could be a second, separate, strand of nucleic acid) which weakly binds to one or both sides of the PTM branch point, polypyrimidine tract, 3′ splice site and/or 5′ splice site (splicing elements), or could bind to parts of the splicing elements themselves. This “safety” binding prevents the splicing elements from being active (i.e. block U2 snRNP or other splicing factors from attaching to the PTM splice site recognition elements). The binding of the “safety” may be disrupted by the binding of the target binding region of the PTM to the target pre-mRNA, thus exposing and activating the PTM splicing elements (making them available to trans-splice into the target pre-mRNA).

The PTMs of the invention will also contain skin cell specific exon sequences, which when trans-spliced to the skin cell specific target pre-mRNA, will result in the formation of a chimeric RNA capable of encoding a functional keratinocyte specific protein. The genomic structure of keratinocyte specific genes such as plectin (Liu CG et al., 1996, Proc. Natl. Acad Sci USA 93:4278-83), Col17A1 (Gatalica B et al., 1997 Am J Hum Genet 60:352-365), type VII collagen (Li, K et al., 1993, Genomics 16:733-9), and laminin (Pulkkinen L et al., 1995 Genomics 25:192-8), to name a few, are known and incorporated herein in their entirety. The specific exon sequences to be included in the structure of the PTM will depend on the specific mutation targeted for correction. Such mutations in the Col17A1 gene include but are not limited to those presented in Table I.

TABLE I
Mutations leading to
preterminal stop codons

| 3781C→G/415insG | (McGrath et al., 1995 Nature Genetics |
|---|---|
| | 11:83-86); |
| 4003delTC/4003delTC | (MGrath et al., 1996 J. Invest Dermatol |
| | 106:771-774); |
| 3514ins25/G627V | (McGrath et al., 1996 Am J. Pathol |
| | 148:1787-96); |
| 4003delTC/Q1403X | (Darling et al., 1997 J. Invest. Dermatol |
| | 108:463-8); |
| 4003delTC/G803X | (Darling et al., 1997 J. Invest. Dermatol |
| | 108:463-8); |
| 2944del5/2944del5 | (Gatalica et al., 1997 Am. J. Hum Genet |
| | 60:352-65); |
| 2944del5/Q1023X | (Gatalica et al., 1997 Am. J. Hum Genet |
| | 60:352-65); |
| 1706delA/R1226X | (Jonkman et al., 1997 Cell 88:543-51); |
| 2342delG2342delG | (Scheffer et al., 1997, Hum Genet |
| | 100:230-5); |
| Q1016X/Q1016X | (Schumann et al., 1997, Am J Hum Genet |
| | 60:1344-53); |
| R1226X/R1226X | (Schumann et al., 1997, Am J Hum Genet |
| | 60:1344-53); |
| 520delAG/520delAG | (Floeth et a1., 1998, J. Invest Dermatol |
| | 111:528-33); |
| 2965delG/2965delG | (Floeth et al., 1998, J. Invest Dermatol |
| | 111:528-33); |
| G539E/2666delTT | (Floeth et al., 1998, J. Invest Dermatol |
| | 111:528-33); |
| G258X/G258X | (Shimizu et al., 1998, J. Invest Dermatol |
| | 111:887-92); |
| 4003delTC/4003delTC | (Darling et al., 1999, J. Clin Invest |
| partially 4080insGG | 103-1371-7); |
| 3781C→T (R1226X) | (Huber et al., 2002, J. Invest Dermatol |
| Ile-18del389 | 118:185-92); |
| R795X/R795X | (Ruzzi et al., 2001 J. Invest Dermatol |
| | 116:182-7) |
| Acceptor splice-site mutations | |
| 2441-2A→G | (Chavanas et al., 1997 J. Invest Dermatol |
| | 109:74-8); |
| 2441-1 G→T/? | (Darling et al., 1998 J. Invest Dermatol |
| | 110:165-9 |
| 3053-1 G→C/3871 + 1 G→C | (Pulkkinen et al., 1999 J. Invest Dermatol |
| | 113:1114-8) |
| Donor splice-site mutation | |
| 3053-1 G→C/3871 + 1 G→C | (Pulkkinen et al., 1999 J. Invest Dermatol |
| | 113:1114-8) |
| 4261 + 1 G→C/4261 + | (van Leusden et al., 2001 81:887-94); |
| 1 G→C | |
| Missense mutations | |
| R1303Q/R1303Q | (Schumann et al. 1997 60:1344-53); |
| G633D/R145X | (Tasanen et al., 2000 J. Invest Dermatol |
| | 115:207-12); and |
| Digenic mutations | |
| L855X/R1226X plus R635X | (Floeth et al., 1999 Am J. Hum Genet |
| (LAMB3 gene) | 65:1530-7). |

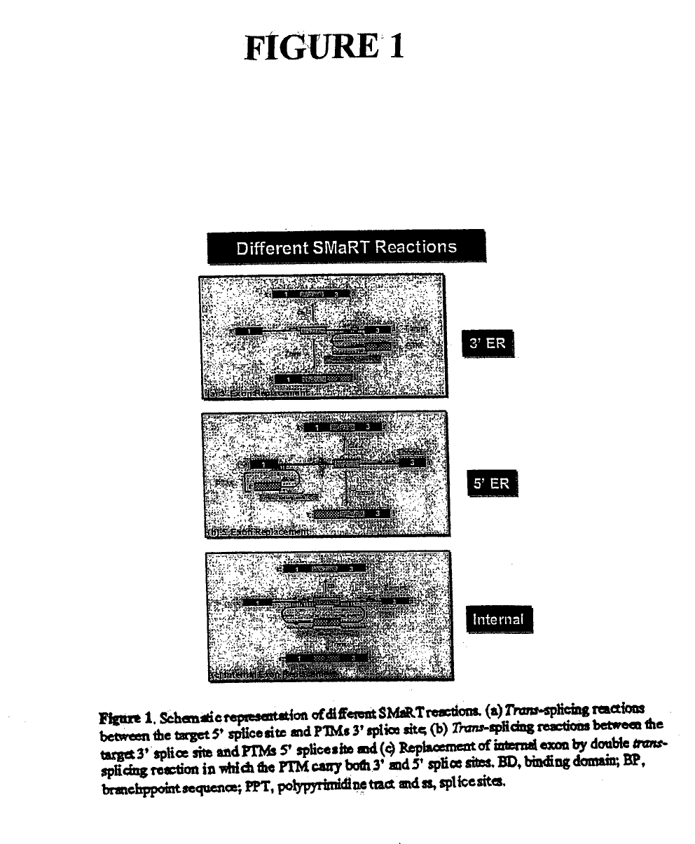

The PTM's of the invention may be engineered to contain a single skin cell specific exon sequence, multiple skin cell specific exon sequences, or alternatively a complete set of skin cell specific exon sequences. The number and identity of the skin cell specific sequences to be used in the PTMs will depend on the targeted specific mutation, and the type of trans-splicing reaction, i.e., 5′ exon replacement, 3′ exon replacement or internal exon replacement that will occur (see FIG. 1). In addition, to limit the size of the PTM, the molecule may include deletions in non-essential regions of skin cell specific target gene. The PTMs may also encode genes useful as markers or imaging reagents, therapeutic genes (toxins, prodrug activating enzymes) etc.

The present invention further provides PTM molecules wherein the coding region of the PTM is engineered to contain mini-introns. The insertion of mini-introns into the coding sequence of the PTM is designed to increase definition of the exon and enhance recognition of the PTM donor site. Mini-intron sequences to be inserted into the coding regions of the PTM include small naturally occurring introns or, alternatively, any intron sequences, including synthetic mini-introns, which include 5′ consensus donor sites and 3′ consensus sequences which include a branch point, a 3′ splice site and in some instances a polypyrimidine tract.

The mini-introns sequences are preferably between about 60-100 nucleotides in length, however, mini-intron sequences of increased lengths may also be used. In a preferred embodiment of the invention, the mini-intron comprises the 5′ and 3′ end of an endogenous intron. In a preferred embodiments of the invention, the 5′ intron fragment is about 20 nucleotides in length and the 3′ end is about 40 nucleotides in length.

In a specific embodiment of the invention, an intron of 528 nucleotides comprising the following sequences may be utilized. Sequence of the intron construct is as follows:

5′ fragment sequence:
gtagttcttttgttcttcactattaagaacttaatttggtgtccatgtctctttttttttctagtttgtagtgctggaag
gtatttttggagaaattcttacatgagcattaggagaatgtatgggtgtagtgtcttgtataatagaaattgttccactgataatttactct
agttttttatttcctcatattattttcagtggctttttcttccacatctttatattttgcaccacattcaacactgtagcggccgc.
3′ fragment sequence:
caactatctgaatcatgtgccccttctctgtgaacctctatcataatacttgtcacactgtattgtaattgtctcttt
tactttcccttgtatcttttgtgcatagcagagtacctgaaacaggaagtattttaaatattttgaatcaaatgagttaatagaatctttac
aaataagaatatacacttctgcttaggatgataattggaggcaagtgaatcctgagcgtgatttgataatgacctaataatgatgggtt
ttatttccag

In yet another specific embodiment of the invention, consensus ISAR sequences are included in the PTMs of the invention (Jones et al., 2001 Nucleic Acid Research 29:3557-3565). Proteins bind to the ISAR splicing activator and repressor consensus sequence which includes a uridine-rich region that is required for 5′ splice site recognition by U1 SnRNP. The 18 nucleotide ISAR consensus sequence comprises the following sequence: GGGCUGAUUUUUCCAUGU. When inserted into the PTMs of the invention, the ISAR consensus sequences are inserted into the structure of the PTM in close proximity to the 5′ donor site of intron sequences. In an embodiment of the invention the ISAR sequences are inserted within 100 nucleotides from the 5′ donor site. In a preferred embodiment of the invention the ISAR sequences are inserted within 50 nucleotides from the 5′ donor site. In a more preferred embodiment of the invention the ISAR sequences are inserted within 20 nucleotides of the 5′ donor site.

The compositions of the invention further comprise PTMs that have been engineered to include cis-acting ribozyme sequences. The inclusion of such sequences is designed to precisely define the length of the PTM by removing any additional or run off PTM transcription. The ribozyme sequences that may be inserted into the PTMs include any sequences that are capable of mediating a cis-acting (self-cleaving) RNA splicing reaction. Such ribozymes include but are not limited to Group I and Group II ribozymes including but not limited to hammerhead, hairpin and hepatitis delta virus ribozymes (see, Chow et al., 1994, J Biol Chem 269:25856-64).

In an embodiment of the invention, splicing enhancers such as, for example, sequences referred to as exonic splicing enhancers may also be included in the structure of the PTMs. Transacting splicing factors, namely the serine/arginine-rich (SR) proteins, have been shown to interact with such exonic splicing enhancers and modulate splicing (See, Tacke et al., 1999, Curr. Opin. Cell Biol. 11:358-362; Tian et al., 2001, J. Biological Chemistry 276:33833-33839; Fu, 1995, RNA 1:663-680). Nuclear localization signals may also be included in the PTM molecule (Dingwell and Laskey, 1986, Ann. Rev. Cell Biol. 2:367-390; Dingwell and Laskey, 1991, Trends in Biochem. Sci. 16:478-481). Such nuclear localization signals can be used to enhance the transport of synthetic PTMs into the nucleus where trans-splicing occurs. In addition, out of reading frame AUG start codons, Kozak sequences or other translational start sites may be included to prevent or minimize PTM self expression.

Additional features can be added to the PTM molecule either after, or before, the nucleotide sequence encoding a translatable protein, such as polyadenylation signals or 5′ splice sequences to enhance splicing, additional binding regions, “safety”-self complementary regions, additional splice sites, or protective groups to modulate the stability of the molecule and prevent degradation.

PTMs may also be generated that require a double-trans-splicing reaction for generation of a chimeric trans-spliced product. Such PTMs could be used to replace an internal exon which could be used for skin cell specific gene repair. PTMs designed to promote two trans-splicing reactions are engineered as described above, however, they contain both 5′ donor sites and 3′ splice acceptor sites. In addition, the PTMs may comprise two or more binding domains and splicer regions. The splicer regions may be placed between the multiple binding domains and two splice sites or alternatively between the multiple binding domains.

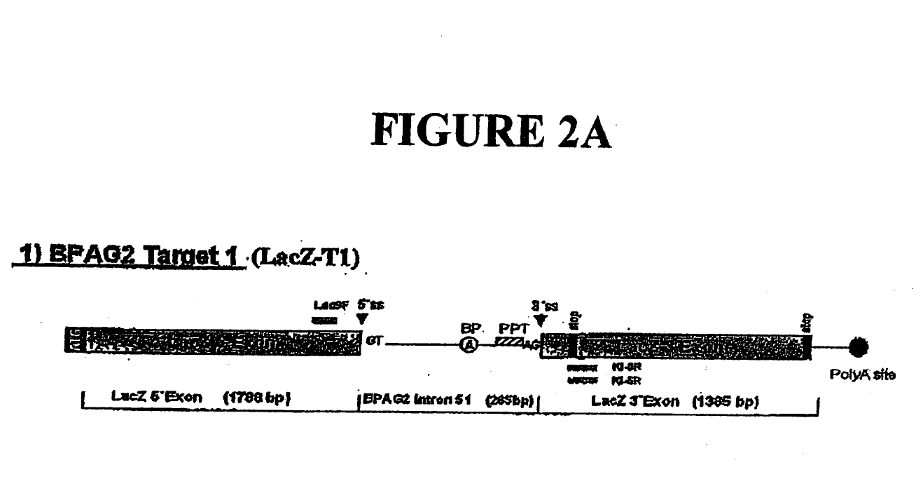

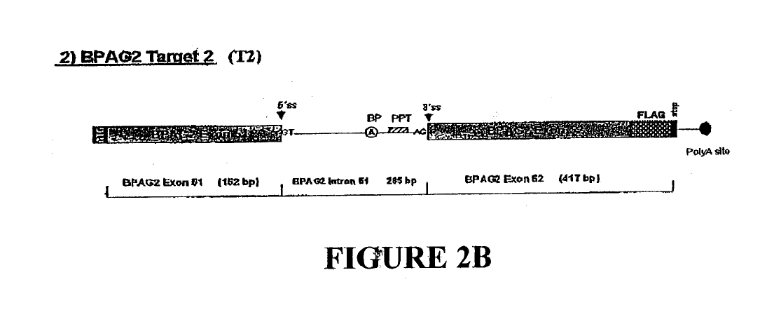

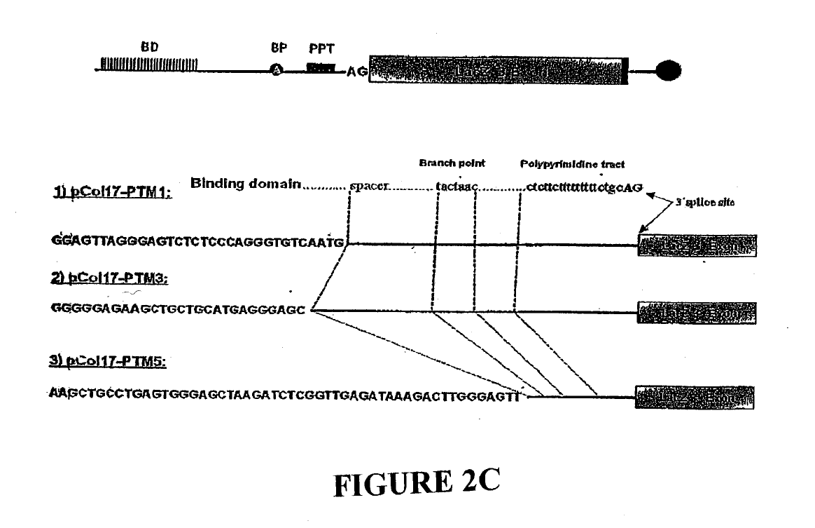

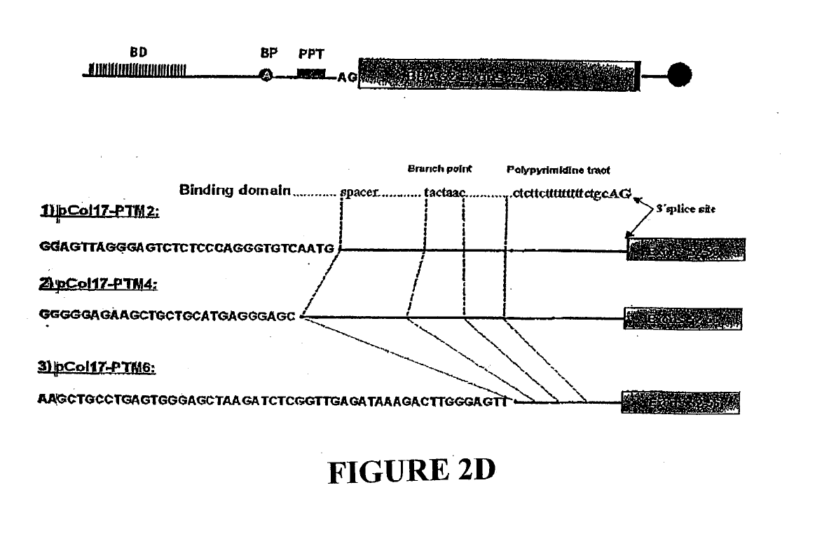

A novel lacZ based assay has been developed for identifying optimal PTM sequences for mediating a desired trans-splicing reaction. The assay permits very rapid and easy testing of many PTMs for their ability to trans-splice. A LacZ keratinocyte specific chimeric target is presented in FIG. 2A. This target consists of the coding region for LacZ (minus 120 nucleotide from the central coding region), split into a 5′ “exon” and a 3′“exon”. Separating these exons is a genomic fragment of the human Col17A1 gene including intron 51. All donor and acceptor sites in this target are functional but a cis-spliced target, which generates a LacZ-keratinocyte specific chimeric mRNA, is non-functional. Trans-splicing between the PTM and target will generate a full length functional LacZ mRNA.

Each new PTM to be tested is transiently co-transfected with the LacZ-keratinocyte specific target using Lipofectamine reagents and then assayed for β-galactosidase activity after 48 hours. Total RNA samples may also be prepared and assessed by RT-PCR using target and PTM specific primers for the presence of correctly spliced repaired products and the level of repaired product. Each trans-splicing domain is engineered with several unique restriction sites, so that when an efficiently spliced sequence is identified based on the analysis of β-gal activity and RT-PCR data, part of or the complete trans-splicing domain, can be readily sub-cloned into a skin cell specific PTM.

When specific PTMs are to be synthesized in vitro (synthetic PTMs), such PTMs can be modified at the base moiety, sugar moiety, or phosphate backbone, for example, to improve stability of the molecule, hybridization to the target specific mRNA, transport into the cell, etc. For example, modification of a PTM to reduce the overall charge can enhance the cellular uptake of the molecule. In addition modifications can be made to reduce susceptibility to nuclease or chemical degradation. The nucleic acid molecules may be synthesized in such a way as to be conjugated to another molecule such as a peptides (e.g., for targeting host cell receptors in vivo), or an agent facilitating transport across the cell membrane (see, e.g., Letsinger et al., 1989, Proc. Natl. Acad. Sci. USA 86:6553-6556; Lemaitre et al., 1987, Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (see, e.g., Krol et al., 1988, BioTechniques 6:958-976) or intercalating agents (see, e.g., Zon, 1988, Pharm. Res. 5:539-549). To this end, the nucleic acid molecules may be conjugated to another molecule, e.g., a peptide, hybridization triggered cross-linking agent, transport agent, hybridization-triggered cleavage agent, etc.

Various other well-known modifications to the nucleic acid molecules can be introduced as a means of increasing intracellular stability and half-life. Possible modifications include, but are not limited to, the addition of flanking sequences of deoxyribonucleotides, peptide nucleic acids and ribonucleotides to the 5′ and/or 3′ ends of the molecule. In some circumstances where increased stability is desired, nucleic acids having modified internucleoside linkages such as 2′-0-methylation may be preferred. Nucleic acids containing modified internucleoside linkages may be synthesized using reagents and methods that are well known in the art (see, Uhlmann et al., 1990, Chem. Rev. 90:543-584; Schneider et al., 1990, Tetrahedron Lett. 31:335 and references cited therein).

The synthetic PTMs of the present invention are preferably modified in such a way as to increase their stability in the cells. Since RNA molecules are sensitive to cleavage by cellular ribonucleases, it may be preferable to use as the competitive inhibitor a chemically modified oligonucleotide (or combination of oligonucleotides) that mimics the action of the RNA binding sequence but is less sensitive to nuclease cleavage. In addition, the synthetic PTMs can be produced as nuclease resistant circular molecules with enhanced stability to prevent degradation by nucleases (Puttaraju et al., 1995, Nucleic Acids Symposium Series No. 33:49-51; Puttaraju et al., 1993, Nucleic Acid Research 21:4253-4258). Other modifications may also be required, for example to enhance binding, to enhance cellular uptake, to improve pharmacology or pharmacokinetics or to improve other pharmaceutically desirable characteristics.

Modifications, which may be made to the structure of the synthetic PTMs include but are not limited to backbone modifications such as use of:

(i) phosphorothioates (X or Y or W or Z=S or any combination of two or more with the remainder as O). e.g., Y═S (Stein, C. A., et al., 1988, Nucleic Acids Res., 16:3209-3221), X═S (Cosstick, R., et al., 1989, Tetrahedron Letters, 30, 4693-4696), Y and Z=S (Brill, W. K.- D., et al., 1989, J. Amer. Chem. Soc., 111:2321-2322); (ii) methylphosphonates (e.g., Z=methyl (Miller, P. S., et al., 1980, J. Biol. Chem., 255:9659-9665); (iii) phosphoramidates (Z=N-(alkyl)2 e.g., alkyl methyl, ethyl, butyl) (Z=morpholine or piperazine) (Agrawal, S., et al., 1988, Proc. Natl. Acad. Sci. USA 85:7079-7083) (X or W═NH) (Mag, M., et al., 1988, Nucleic Acids Res., 16:3525-3543); (iv) phosphotriesters (Z=O-alkyl e.g., methyl, ethyl, etc) (Miller, P. S., et al., 1982, Biochemistry, 21:5468-5474); and (v) phosphorus-free linkages (e.g., carbamate, acetamidate, acetate) (Gait, M. J., et al., 1974, J. Chem. Soc. Perkin I, 1684-1686; Gait, M. J., et al., 1979, J. Chem. Soc. Perkin I, 1389-1394).

In addition, sugar modifications may be incorporated into the PTMs of the invention. Such modifications include the use of: (i) 2′-ribonucleosides (R=H); (ii) 2′-O-methylated nucleosides (R═OMe)) (Sproat, B. S., et al., 1989, Nucleic Acids Res., 17:3373-3386); and (iii) 2′-fluoro-2′-riboxynucleosides (R═F) (Krug, A., et al., 1989, Nucleosides and Nucleotides, 8:1473-1483).

Further, base modifications that may be made to the PTMs, including but not limited to use of: (i) pyrimidine derivatives substituted in the 5-position (e.g., methyl, bromo, fluoro etc) or replacing a carbonyl group by an amino group (Piccirilli, J. A., et al., 1990, Nature, 343:33-37); (ii) purine derivatives lacking specific nitrogen atoms (e.g., 7-deaza adenine, hypoxanthine) or functionalized in the 8-position (e.g., 8-azido adenine, 8-bromo adenine) (for a review see Jones, A. S., 1979, Int. J. Biolog. Macromolecules, 1:194-207).

In addition, the PTMs may be covalently linked to reactive functional groups, such as: (i) psoralens (Miller, P. S., et al., 1988, Nucleic Acids Res., Special Pub. No. 20, 113-114), phenanthrolines (Sun, J- S., et al., 1988, Biochemistry, 27:6039-6045), mustards (Vlassov, V. V., et al., 1988, Gene, 72:313-322) (irreversible cross-linking agents with or without the need for co-reagents); (ii) acridine (intercalating agents) (Helene, C., et al., 1985, Biochimie, 67:777-783); (iii) thiol derivatives (reversible disulphide formation with proteins) (Connolly, B. A., and Newman, P. C., 1989, Nucleic Acids Res., 17:4957-4974); (iv) aldehydes (Schiff's base formation); (v) azido, bromo groups (UV cross-linking); or (vi) ellipticines (photolytic cross-linking) (Perrouault, L., et al., 1990, Nature, 344:358-360).

In an embodiment of the invention, oligonucleotide mimetics in which the sugar and internucleoside linkage, i.e., the backbone of the nucleotide units, are replaced with novel groups can be used. For example, one such oligonucleotide mimetic which has been shown to bind with a higher affinity to DNA and RNA than natural oligonucleotides is referred to as a peptide nucleic acid (PNA) (for review see, Uhlmann, E. 1998, Biol. Chem. 379:1045-52). Thus, PNA may be incorporated into synthetic PTMs to increase their stability and/or binding affinity for the target pre-mRNA.

In another embodiment of the invention synthetic PTMs may covalently linked to lipophilic groups or other reagents capable of improving uptake by cells. For example, the PTM molecules may be covalently linked to: (i) cholesterol (Letsinger, R. L., et al., 1989, Proc. Natl. Acad. Sci. USA, 86:6553-6556); (ii) polyamines (Lemaitre, M., et al., 1987, Proc. Natl. Acad. Sci, USA, 84:648-652); other soluble polymers (e.g., polyethylene glycol) to improve the efficiently with which the PTMs are delivered to a cell. In addition, combinations of the above identified modifications may be utilized to increase the stability and delivery of PTMs into the target cell.

The PTMs of the invention can be used in methods designed to produce a novel chimeric RNA in a target cell so as to result in correction of skin cell specific genetic defects. The methods of the present invention comprise delivering to a skin cell a PTM which may be in any form used by one skilled in the art, for example, an RNA molecule, or a DNA vector which is transcribed into a RNA molecule, wherein said PTM binds to a skin cell specific pre-mRNA and mediates a trans-splicing reaction resulting in formation of a chimeric RNA comprising a portion of the PTM molecule spliced to a portion of the pre-mRNA.

### 5.2 Synthesis of the Trans-Splicing Molecules

The nucleic acid molecules of the invention can be RNA or DNA or derivatives or modified versions thereof, single-stranded or double-stranded. By nucleic acid is meant a PTM molecule or a nucleic acid molecule encoding a PTM molecule, whether composed of deoxyribonucleotides or ribonucleosides, and whether composed of phosphodiester linkages or modified linkages. The term nucleic acid also specifically includes nucleic acids composed of bases other than the five biologically occurring bases (adenine, guanine, thymine, cytosine and uracil). In addition, the PTMs of the invention may comprise, DNA/RNA, RNA/protein or DNA/RNA/protein chimeric molecules that are designed to enhance the stability of the PTMs.

The PTMs of the invention can be prepared by any method known in the art for the synthesis of nucleic acid molecules. For example, the nucleic acids may be chemically synthesized using commercially available reagents and synthesizers by methods that are well known in the art (see, e.g., Gait, 1985, Oligonucleotide Synthesis: A Practical Approach, IRL Press, Oxford, England).

Alternatively, synthetic PTMs can be generated by in vitro transcription of DNA sequences encoding the PTM of interest. Such DNA sequences can be incorporated de variety of vectors downstream from suitable RNA polymerase promoters such as the T7, SP6, or T3 polymerase promoters. Consensus RNA polymerase promoter sequences include the following:

| | |
|---|---|
| T7: | TAATACGACTCACTATAGGGAGA |
| SP6: | ATTTAGGTGACACTATAGAAGNG |
| T3: | AATTAACCCTCACTAAAGGGAGA. |

The base in bold is the first base incorporated into RNA during transcription. The underline indicates the minimum sequence required for efficient transcription.

RNAs may be produced in high yield via in vitro transcription using plasmids such as SPS65 and Bluescript (Promega Corporation, Madison, Wis.). In addition, RNA amplification methods such as Q-β amplification can be utilized to produce the PTM of interest.

The PTMs may be purified by any suitable means, as are well known in the art. For example, the PTMs can be purified by gel filtration, affinity or antibody interactions, reverse phase chromatography or gel electrophoresis. Of course, the skilled artisan will recognize that the method of purification will depend in part on the size, charge and shape of the nucleic acid to be purified.

The PTM's of the invention, whether synthesized chemically, in vitro, or in vivo, can be synthesized in the presence of modified or substituted nucleotides to increase stability, uptake or binding of the PTM to a target pre-mRNA. In addition, following synthesis of the PTM, the PTMs may be modified with peptides, chemical agents, antibodies, or nucleic acid molecules, for example, to enhance the physical properties of the PTM molecules. Such modifications are well known to those of skill in the art.

In instances where a nucleic acid molecule encoding a PTM is utilized, cloning techniques known in the art may be used for cloning of the nucleic acid molecule into an expression vector. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

The DNA encoding the PTM of interest may be recombinantly engineered into a variety of host vector systems that also provide for replication of the DNA in large scale and contain the necessary elements for directing the transcription of the PTM. The use of such a construct to transfect target cells in the patient will result in the transcription of sufficient amounts of PTMs that will form complementary base pairs with the endogenously expressed cell specific pre-mRNA targets and thereby facilitate a trans-splicing reaction between the complexed nucleic acid molecules. For example, a vector can be introduced in vivo such that it is taken up by a cell and directs the transcription of the PTM molecule. Such a vector can remain episomal or become chromosomally integrated, as long as it can be transcribed to produce the desired RNA, i.e., PTM. Such vectors can be constructed by recombinant DNA technology methods standard in the art.

Vectors encoding the PTM of interest can be plasmid, viral, or others known in the art, used for replication and expression in mammalian cells. Expression of the sequence encoding the PTM can be regulated by any promoter/enhancer sequences known in the art to act in mammalian, preferably human cells. Such promoters/enhancers can be inducible or constitutive. Such promoters include but are not limited to: the SV40 early promoter region (Benoist, C. and Chambon, P. 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), the viral CMV promoter, the human β-chorionic gonadotropin-6 promoter (Hollenberg et al., 1994, Mol. Cell. Endocrinology 106:111-119), etc. In a preferred embodiment of the invention, keratinocyte specific promoter/enhancer sequences may be used to promote the synthesis of PTMs in keratinocytes. Such promoters include, for example, the keratin 14 promoter which targets gene expression to the basal layer of the epidermis (Wang X et al., 1997, Proc Natl. Acad Sci 94:219-26), the loricrin promoter (Disepio et al., 1995, J. Biol Chem 270:10792-9) which targets expression to the upper layers of the epidermis and the involucrin promoter transcriptional response element (Phillips et al., 2000, Biochem. J. 348:45-53).

Vectors for use in the practice of the invention include any eukaryotic expression vectors, including but not limited to viral expression vectors such as those derived from the class of retroviruses, adenoviruses or adeno-associated viruses.

### 5.3 Uses and Administration of Trans-Splicing Molecules

The compositions and methods of the present invention can be utilized to correct skin cell specific genetic defects. Specifically, targeted trans-splicing, including double-trans-splicing reactions, 3′ exon replacement and/or 5′ exon replacement can be used to repair or correct skin cell specific transcripts that are either truncated or contain mutations. The PTMs of the invention are designed to cleave a targeted transcript upstream or downstream of a specific mutation or upstream of a premature 3′ stop termination and correct the mutant transcript via a trans-splicing reaction which replaces the portion of the transcript containing the mutation with a functional sequence.

Various delivery systems are known and can be used to transfer the compositions of the invention into cells, e.g., encapsulation in liposomes, microparticles, microcapsules, recombinant cells capable of expressing the composition, receptor-mediated endocytosis (see, e.g., Wu and Wu, 1987, J. Biol. Chem. 262:4429-4432), construction of a nucleic acid as part of a retroviral, adenoviral, adeno-associated viral or other vector, injection of DNA, electroporation, calcium phosphate mediated transfection, etc.

The compositions and methods can be used to provide sequences encoding a functional biologically active skin cell specific molecule to cells of an individual with an inherited genetic disorder or other type of skin disorder where expression of the missing or mutant gene product produces a normal phenotype. In addition, the compositions and methods of the invention can be used to inhibit the proliferation of cells of the skin in an individual with cancer of the skin or psoriasis, for example. In such instances the PTMs may be designed to interact with target pre-mRNAs that encode regulators of skin cell proliferation and inhibit the expression of such regulators and encodes a reporter molecule.

In a preferred embodiment, nucleic acids comprising a sequence encoding a PTM are administered to promote PTM function, by way of gene delivery and expression into a host cell. In this embodiment of the invention, the nucleic acid mediates an effect by promoting PTM production. Any of the methods for gene delivery into a host cell available in the art can be used according to the present invention. For general reviews of the methods of gene delivery see Strauss, M. and Barranger, J. A., 1997, Concepts in Gene Therapy, by Walter de Gruyter & Co., Berlin; Goldspiel et al., 1993, Clinical Pharmacy 12:488-505; Wu and Wu, 1991, Biotherapy 3:87-95; Tolstoshev, 1993, Ann. Rev. Pharmacol. Toxicol. 33:573-596; Mulligan, 1993, Science 260:926-932; and Morgan and Anderson, 1993, Ann. Rev. Biochem. 62:191-217; 1993, TIBTECH 11(5):155-215. Exemplary methods are described below.

Delivery of the PTM into a host cell may be either direct, in which case the host is directly exposed to the PTM or PTM encoding nucleic acid molecule, or indirect, in which case, host cells are first transformed with the PTM or PTM encoding nucleic acid molecule in vitro, then transplanted into the host. These two approaches are known, respectively, as in vivo or ex vivo gene delivery.

In a specific embodiment, the nucleic acid is directly administered in vivo, where it is expressed to produce the PTM. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e.g., Wu and Wu, 1987, J. Biol. Chem. 262:4429-4432).

In a specific embodiment, a viral vector that contains the PTM can be used. For example, a retroviral vector can be utilized that has been modified to delete retroviral sequences that are not necessary for packaging of the viral genome and integration into host cell DNA (see Miller et al., 1993, Meth. Enzymol. 217:581-599). Alternatively, adenoviral or adeno-associated viral vectors can be used for gene delivery to cells or tissues. (See, Kozarsky and Wilson, 1993, Current Opinion in Genetics and Development 3:499-503 for a review of adenovirus-based gene delivery).

In a preferred embodiment of the invention an adeno-associated viral vector may be used to deliver nucleic acid molecules capable of encoding the PTM. The vector is designed so that, depending on the level of expression desired, the promoter and/or enhancer element of choice may be inserted into the vector.

Another approach to gene delivery into a cell involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. The resulting recombinant cells can be delivered to a host by various methods known in the art. In a preferred embodiment, the cell used for gene delivery is autologous to the host cell.

In a specific embodiment of the invention, skin cells, such as keratinocytes, may be removed from a subject having a skin disorder and transfected with a nucleic acid molecule capable of encoding a PTM designed to correct a skin cell specific disorder such as a genetic disorder. Cells may be further selected, using routine methods known to those of skill in the art, for integration of the nucleic acid molecule into the genome thereby providing a stable cell line expressing the PTM of interest. Such cells are then transplanted into the subject thereby providing a source of skin cell specific protein.

The present invention also provides for pharmaceutical compositions comprising an effective amount of a PTM or a nucleic acid encoding a PTM, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Examples of suitable pharmaceutical carriers are described in “Remington's Pharmaceutical sciences” by E. W. Martin.

In specific embodiments, pharmaceutical compositions are administered in diseases or disorders involving an absence or decreased (relative to normal or desired) level of an endogenous skin cell specific protein or function, for example, in hosts where the skin cell specific protein is lacking, genetically defective, biologically inactive or underactive, or under expressed. Such disorders include but are not limited to epidermal fragility disorders, keratinization disorders, hair disorders, pigmentation disorders, prophyrias, pre-cancerous and cancer disorders. In addition, pharmaceutical compositions may be administered in proliferative disorders of the skin, such as cancers and psoriasis, where the goal is to inhibit the proliferation of such cells. The activity of the skin cell specific protein encoded for by the chimeric mRNA resulting from the PTM mediated trans-splicing reaction can be readily detected, e.g., by obtaining a host tissue sample (e.g., from biopsy tissue) and assaying it in vitro for mRNA or protein levels, structure and/or activity of the expressed chimeric mRNA. Many methods standard in the art can be thus employed, including but not limited to immunoassays to detect and/or visualize the protein encoded for by the chimeric mRNA (e.g., Western blot, immunoprecipitation followed by sodium dodecyl sulfate polyacrylamide gel electrophoresis, immunocytochemistry, etc.) and/or hybridization assays to detect formation of chimeric mRNA expression by detecting and/or visualizing the presence of chimeric mRNA (e.g., Northern assays, dot blots, in situ hybridization, and reverse-transcription PCR, etc.), etc.

In a specific embodiment, it may be desirable to administer the pharmaceutical compositions of the invention locally to the area in need of treatment, i.e., skin. This may be achieved by, for example, and not by way of limitation, topical application, e.g., in conjunction with a wound dressing after surgery, by injection, or by means of an implant, said implant being of a porous, non-porous, or gelatinous material, including membranes, such as sialastic membranes, or fibers. Other control release drug delivery systems, such as nanoparticles, matrices such as controlled-release polymers, hydrogels.

The PTM will be administered in amounts which are effective to produce the desired effect in the targeted cell. Effective dosages of the PTMs can be determined through procedures well known to those in the art which address such parameters as biological half-life, bioavailability and toxicity. The amount of the composition of the invention which will be effective will depend on the severity of the skin disorder being treated, and can be determined by standard clinical techniques. Such techniques include analysis of skin samples to determine levels of protein expression. In addition, in vitro assays may optionally be employed to help identify optimal dosage ranges.

The present invention also provides a pharmaceutical pack or kit comprising one or more containers filled with one or more of the ingredients of the pharmaceutical compositions of the invention optionally associated with such container(s) can be a notice in the form prescribed by a governmental agency regulating the manufacture, use or sale of pharmaceuticals or biological products, which notice reflects approval by the agency of manufacture, use or sale for human administration.

### 6. EXAMPLE

### Trans-Splicing of the COL17A1 Gene

The data presented below demonstrates the feasibility of using trans-splicing reactions in a keratinocyte specific context for skin gene therapy. In particular, the data indicates that (i) the trans-splicing reaction is accurate between the target and PTM in keratinocytes; (ii) effectivity can be modulated by incorporating stem-loop structures in the trans-splicing domain; and (iii) intron 51 of the Col17A1 gene can be targeted and trans-spliced using spliceosomal mediated trans-splicing at the pre-mRNA level in keratinocytes.

### 6.1 Materials and Methods

Cell culture. Human embryonic kidney cells (293T) were grown at 37° C. and 5% CO₂ in a humidified incubator in DMEM medium supplemented with 10% FBS (Life Technologies, Gaithersburg, Md.). Passaging of the cells was performed every 3-4 days using 1% Trypsin-EDTA (PAA Laboratories, Linz, Austria) and cells were replated at the desired density. Human keratinocytes used in all experiments were prepared from neonatal foreskins using a standard protocol. Cells were counted and plated on 60 mm plates at the desired density and grown for 10-12 days at 37° C. and 5% CO₂ in a humidified incubator in KGM-2 medium (Clonetics/Bio-Whittaker, Walkersville, MD) to a confluency of approximately 50-60%. Medium was changed every 2-3 days.

Primary keratinocytes from a GABEB patient homozygous for 4003delTC in COL17A1 were immortalized with a human papilloma virus HPV16 E6 and E7 vector and continuously passaged; the resulting cell line did not express collagen XVII protein. Cells were maintained at 37° C. and 5% CO₂ in KGM-2 medium (Clonetics) and passaged every 5-7 days at a confluency of approximately 70% and replated at the desired density. Medium was changed every 2-3 days.

Target construction. LacZ-T1 (FIG. 2A) included a lacZ 5′ “exon” (1-1788 bp) followed by intron 51 of the Collagen 17A1 gene (282 bp) and a LacZ 3′ “exon” (1789-3174 bp). This lacZ 3′ exon contained two stop codons at position 1800 bp. Intron 51 of Col17A1 was amplified by PCR with Pfu DNA polymerase (Stratagene, La Jolla, Calif.) using genomic DNA as template and primers:

| | |
|---|---|
| Int51U | (5′-CGGGATCCGTAGGTGCCCCGACGGTGATG-3′); and |
| Int51D | (5′CTAGGGTAACCAGGGTGAGAAGCTGCATGAGT-3′). |

The amplified product was digested with BamHI and BstEII (New England Biolabs, Beverly, Mass.) and inserted between the two lacZ exons. T2 (FIG. 2B) included the genomic sequence of exon 51, intron 51 and exon 52 followed by a FLAG sequence. The genomic sequence of exon 51, intron 51 and exon 52 was amplified using Pfu DNA polymerase and primers:

| | |
|---|---|
| COLI-F | (5′-CTAGGCTAGCCTGCCGGCTTGTCATTCATCC-3′) and |
| COLI-R | (5′-CTAGAAGCTTTTACTTGTCATCGTCGTCCTTGTA |
| | GTCGCTGCATGCTCTCTGACACC-3′). |

The FLAG sequence was introduced by primer COLI-R. The PCR product was digested with NheI and HindIII (New England Biolabs, Beverly, Mass.) and inserted in pcDNA3.1 (Invitrogen, Carlsbad, Calif.).

Pre-trans-splicing molecules (PTMs). pCOL17-PTM1 (FIG. 2C) was constructed by digesting PTM14 (Intronn Inc., Rockville, Md.) with EcoRI and KpnI replacing the CFTR binding domain (Mansfield S G et al., 2000 7:1885-95) with a 80 bp oligonucleotide containing a 32 bp antisense binding domain (BD), a 18 bp spacer, branch point (BP), a polypyrimidine tract (PPT), and an acceptor AG dinucleotide followed by a lacZ 3′ exon (1789-3174 bp). The use of BP and PPT follows consensus sequences which are needed for performance of the two phosphoryl transfer reactions involved in cis-splicing and also in trans-splicing, pCOL17-PTM4 and pCOL17-PTM6 were constructed by digesting PTM1, 3, and 5 with KpnI and HindIII and replacing the lacZ 3′ exon with the exon 52 to 56 cDNA sequence of COLI7A1 (FIG. 2D). The cDNA sequence was amplified with Pfu DNA polymerase from poly-dT primed cDNA using the following primers:

| | |
|---|---|
| COL2-F | (5′-CTAGGGTACCTCTTCTTTTTTTTGATATCCTGCA |
| | GGTCCTGATGTGCGCAGC-3′); and |
| COL-2-R | (5′-CTAGAAGCTTTTATGGAGACCTTGGACCTAAG-3′). |

The amplified product was digested with KpnI and HindIII and cloned into PTMs 1, 3, and 5. All constructs were sequenced to confirm their correct sequence.

Transfection into 293T cells. 293T cells were used for preliminary experiments due to their lack of endogenous COLI7AI mRNA. The day before transfection, 1.15×10⁶ cells were plated on 60 mm plates and grown for 24 hr. Cells were transfected with expression plasmids using LipofectaminePlus reagent (Life Technologies) according to manufacturer's protocol. Cells were harvested 48 hr after transfection.

Transfection into primary keratinocytes (hKC). hKC were grown as described above for 10-12 days to a confluency of 50-60%. Cells were transfected using LipofectaminePlus reagent and KGM-2 without supplements. KGM-2 medium containing 2× supplements was added 3 hours after transfection. The medium was replaced by regular KGM-2 after 12 hr and incubated for additional 24-48 hr at 37° C.

Transfection into immortalized GABEB-keratinocytes. GABEB keratinocytes were plated on 60 mm diameter plates at a density of 1×10⁶ cells/ml and grown to 60-70% confluency. Cells were transfected with FuGENE 6 (Roche) transfection reagent (6 μl/μg DNA) and DNA in supplement-free KGM-2 medium according to the manufacturer's protocol. The transfection reaction was added dropwise to the cells and incubated for 3 hr at 37° C. in 5% CO₂. Then KGM-2 with 2× supplements was added and the incubation was continued overnight. The next morning the medium was replaced with fresh medium and incubated for additional 24-48 hr.

Total RNA isolation. 48 hr after transfection the plates were rinsed with phosphate buffered saline (PBS) once and the cells were harvested in 1 ml PBS. The cells were pelleted and the supernatant was removed. Total RNA was isolated using MasterPure RNA/DNA purification kit (Epicentre Technologies, Madison, Wis.). Contaminating DNA was removed by DNase I treatment for 30-60 min at 37° C.

Reverse Transcription Polymerase Chain Reaction. RT-PCR was performed using a SuperScript OneStep™ RT-PCR Kit (Life Technologies) according to the manufacturer's protocol. Each reaction contained 50 to 500 ng of total RNA and 100 ng of a 5′- and 3′-specific primer in a 25 μl reaction volume. RT-PCR products were separated by gel-electrophoresis using 2% agarose gels. Primers used to estimate the products of cis and trans-splicing were as follows:

LAC9F (5′-ATCAAATCTGTCGATCCTTCC-3′); and

KI-3R (5′-GACTGATCCACCCAGTCCCATTA-3′) for cis-, and LAC9F and

KI-5R (5′-GACTGATCCACCCAGTCCCAGAC-3′) for trans-splicing.

For position of these primers on the plasmids see FIG. 2A. RT-PCR analysis for the COLI7A1-mini-gene cis-splicing was performed using the following primers:

Ex51-1F (5′-CATCCCAGGCCCTCCAGGAC-3′); and

FLAG-R (5′-TTGTCATCGTCGTCCTTGTAG-3′), while Primers Ex51-1F and

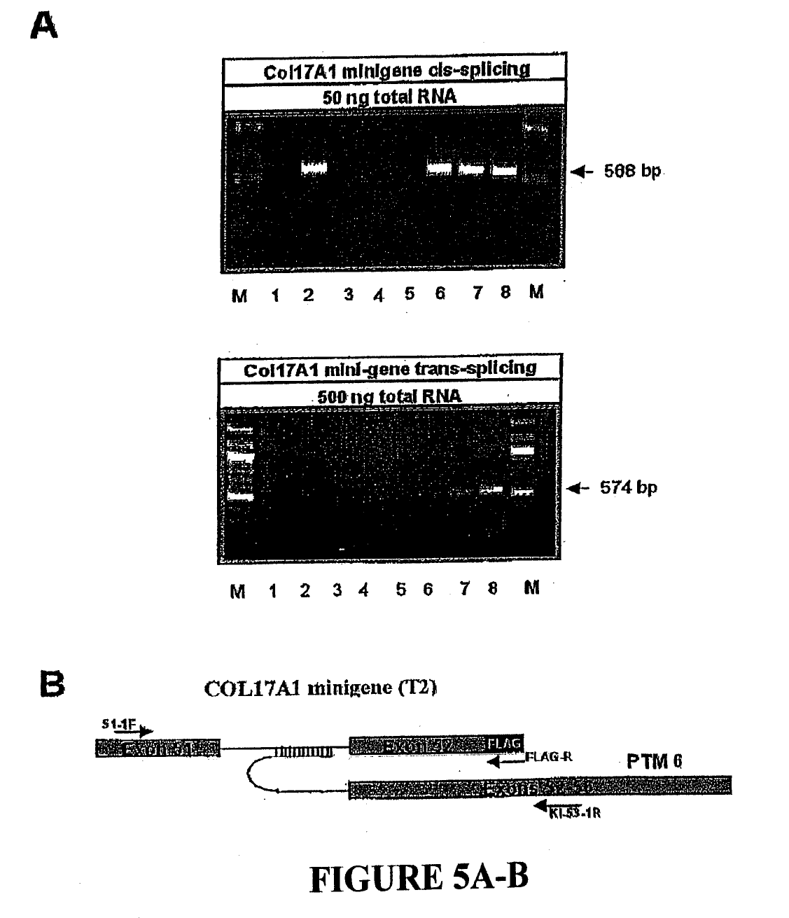

KI-53-1R (5′-GTAGGCCATCCCTTGCAG-3′) were used for the detection of trans-splicing. For position of these primers on the plasmids see FIG. 5B.

Protein preparation and β-gal assay. The total protein from transfected cells was isolated by a freeze and thaw method and assayed for β-gal activity as described (Invitrogen). Total protein concentration was measured by the dye-binding assay according to Bradford using Bio-Rad protein assay reagent (BIO-RAD, Hercules, Calif.). All measurements of protein concentrations and β-gal activities were performed with a Pharmacia Ultrospec 2000 Spectrophotometer (Amersham Pharmacia, Uppsala, Sweden).

In situ staining for β-gal. The expression of functional β-gal was monitored using a β-gal staining kit (Invitrogen) following the manufacturer's protocol. The percentage of β-gal positive cells was determined by counting stained versus unstained cells in five randomly selected fields.

DNA sequencing. Constructs and RT-PCR products were sequenced using an ABI Prism automated sequencer (Applied Biosystems, Foster City, Calif.), Taq Dideoxy Terminator Cycle Sequencing Kit (Applied Biosystems), and 2 pmol of primer per reaction to verify sequences.

RNA structure determination. RNA secondary structures for PTM binding domain design were predicted using the RNA folding program mfold by Zucker and Turner (http://mfold2.wustl.edu/˜mfold/ma/formI.cgi).

Quantitative real-time RT-PCR analysis. Real-time RT-PCR was performed using a LightCycler (Roche Diagnostics, Mannheim, Germany). 1 μg total RNA obtained from the transfection experiments was oligo dT primed and reversed transcribed to cDNA using M-MLV-RT (Promega, Madison, Wis.). PCR reactions were performed according to the manufacturers protocol using 1 μl of the cDNA solution, 3 μl SYBR-green master mix (Roche), 2 pmol of primer Lac9F, 2 pmol of primer KI-3R for the cis-splicing, and 2 pmol of Primer KI-5R for the trans-splicing product.

### 6.2 Results

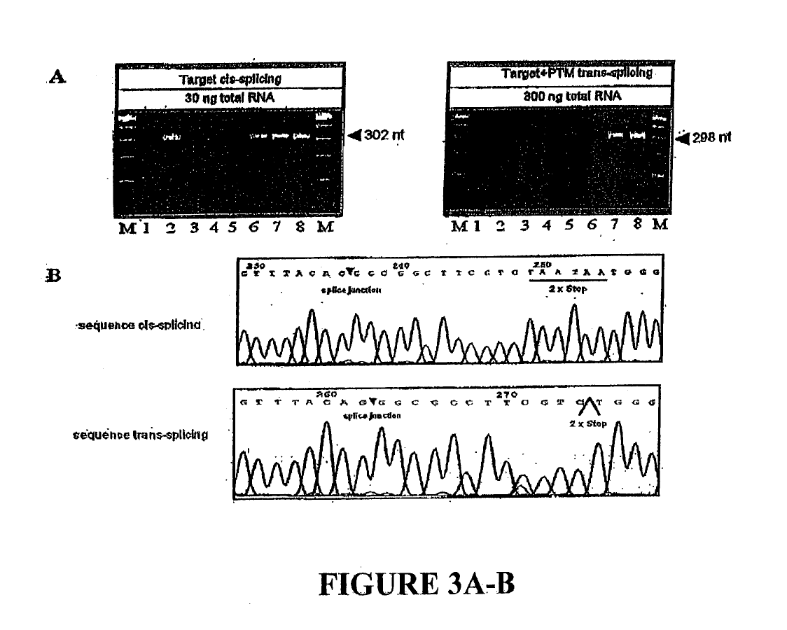

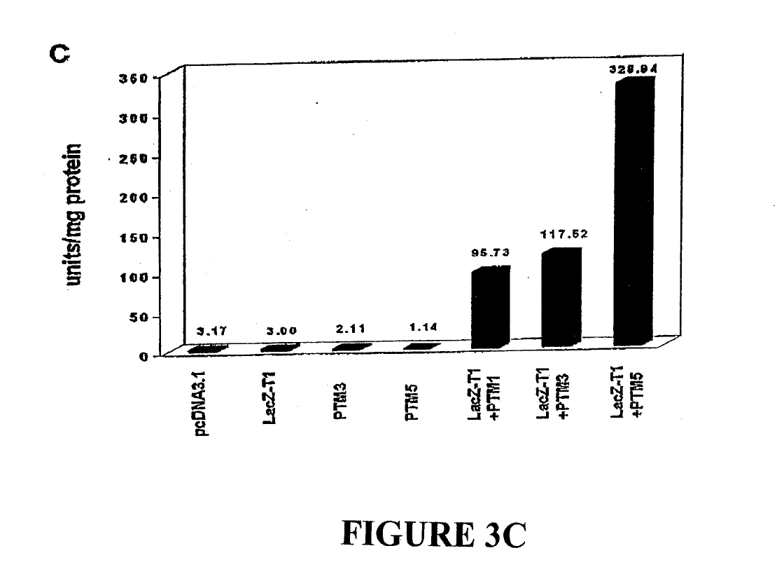

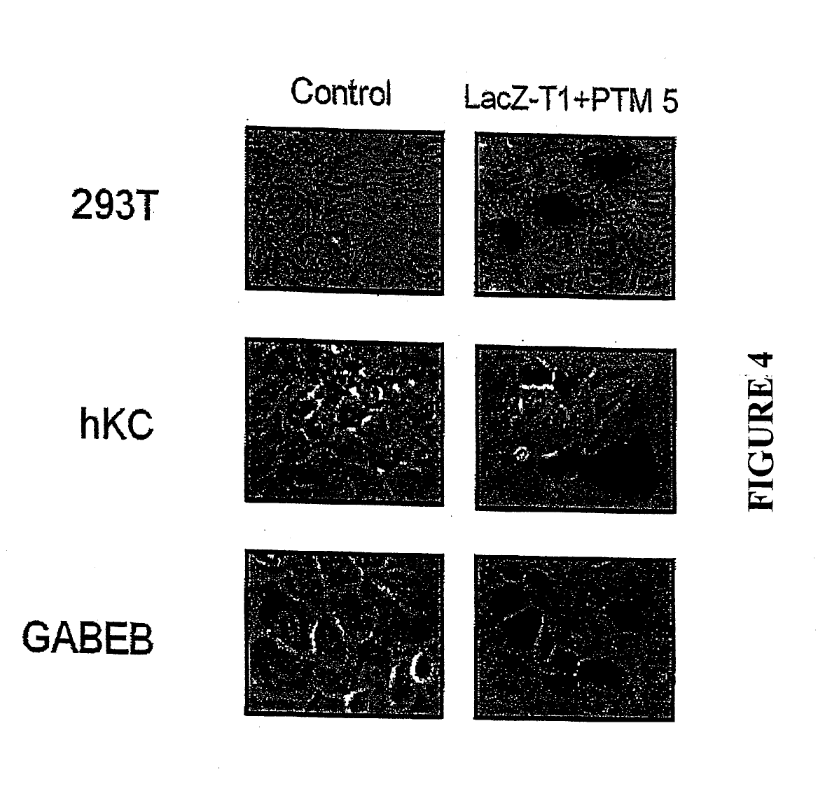

The LacZ model repair-system. To evaluate the efficiency of trans-splicing in various cell types we used a lacZ model repair-system. It consists of a mutant β-gal target expressed from plasmid LacZ-T1, and a second plasmid expressing a pre-trans-splicing molecule (PTM) which were cotransfected into the respective cells. First, cis-splicing was examined by transfecting LacZ-T1 plasmid (FIG. 2A) into 293T cells followed by preparation of total RNA and RT-PCR analysis. A 302 bp RT-PCR product was detected using primers Lac9F (lacZ 5′exon specific) and KI-3R (LacZ 3′Stop codon specific), demonstrating the expected size for accurate cis-splicing (FIG. 3A; lanes 2; 6; 7; 8). The RT-PCR product was sequenced to confirm the accuracy of splice site usage (FIG. 3B, upper panel). Because of the inclusion of in-frame stop codons there is no measurable β-gal activity exceeding basal expression of mock transfections (FIG. 3C). 293T cells transfected with LacZ-T1 alone showed complete absence of positively stained cells in cell culture (FIG. 4, top panel, control).

The second component of the lacZ model repair-system are the PTMs. PTM1 included a 32 bp antisense binding domain exactly complementary to the 3′ end of COL17A1 intron 51, and 18 bp spacer sequence, yeast branch point (BP), polypyrimidine tract (PPT) and a 3′ splice acceptor site followed by the coding sequence of the wild-type lacZ gene fragment from nucleotide 1789 to 3174 inserted into a pcDNA3.1 mammalian expression vector (FIG. 2C). This construct was predicted to produce RNA which binds to and repairs the defective pre-mRNA transcribed from LacZ-T1 by replacing the mutation in the 3′exon of the target pre-mRNA and therefore restoring β-gal activity. As expected the PTM did not yield functional mRNA (FIG. 3A; lanes 3, 4, 5; left picture) and β-gal activity (FIG. 3C) when transfected alone.

Testing for RNA repair and protein function restoration in an epithelia cell-line. The ability of PTM-induced RNA trans-splicing to repair the chosen pre-mRNA target was examined in a transient co-transfection assay. Plasmids expressing LacZ-T1 pre-mRNA and PTM1 pre-mRNA were co-transfected into 293T cells. The product of the trans-splicing reaction should be an mRNA consisting of the 5′exon of lacZ and the inserted normal 3′exon of lacZ, which should be translated into functional β-gal protein. Analysis of total RNA by RT-PCR using a target specific primer (Lac9F) and a lacZ-PTM specific primer (KI-5R) showed a RT-PCR product of the predicted length (298 bp) (FIG. 3A; lanes 6, 7, 8; right picture). This product was not observed in cells transfected with either LacZ-T1 or PTM1 alone (FIG. 3A; lanes 2, 3, 4, 5; right picture). Sequencing of the 298 bp trans-spliced RT-PCR product demonstrated that trans-splicing was accurate between LacZ-T1 pre-mRNA and PTM1 pre-mRNA (FIG. 3B; lower panel). In addition, genomic DNA was prepared from co-transfected cells and analyzed by PCR using the target specific Lac9F as a forward and the PTM specific KI-5R as a reverse primer to rule out recombination events on the DNA level between PTM and target. No PCR fragment was detected indicating the absence of recombination events.

Trans-splicing between LacZ-T1 pre-mRNA and PTM1 pre-mRNA restores β-gal activity. The repair of defective lacZ pre-mRNA by trans-splicing and production of functional β-gal protein was investigated in 293T cells co-transfected with target and PTM plasmids. Staining of co-transfected 293T cells revealed β-gal positive cells (25% of total cells) (FIG. 4 upper panel, right), indicating the production of corrected RNA. In contrast, cells transfected with either LacZ-T1 or PTM1 alone did not produce any functional β-gal as indicated by the complete absence of β-gal positive cells.

To further quantify the amount of β-gal activity produced by trans-splicing repair enzyme activity was measured in a colorometric assay. β-gal activity in protein extracts prepared from cells transfected with either LacZ-T1 target or PTM1 alone was almost identical to the background levels. In contract, cells co-transfected with LacZ-T1 and PTM1 produced a significant amount of β-gal activity compared to background (˜100 fold increase) (FIG. 3C). These data demonstrate the efficient repair of defective LacZ-T1 pre-mRNA by trans-splicing restoring β-gal protein function.

The length of the binding domains can modulate trans-splicing efficiency and specificity. To determine how the length of the binding domains influences the efficiency of trans-splicing between LacZ-T1 and PTMs, PTM3 and PTM5 were constructed (FIG. 2C). PTM3 contains a shorter binding of 25 nt with distinct changes in the nucleotide sequence to achieve a tight RNA secondary structure that should reduce non-specific binding to other RNA targets. This change was made based on the predictions gained from the RNA program of Zucker and Turner http://mfold2.wustl.edu/˜mfold/ma/formI.cgi). This PTM (PTM3) was co-transfected with LacZ-T1 and its repair efficiency was measured by RT-PCR, β-gal quantitative assay and in situ staining for β-gal. PTM3 showed a modest increase in β-gal activity compared to PTM1 indicating more efficient binding and trans-splicing. A third PTM, PTM5 was constructed using a longer binding domain of 52 nt (FIG. 2C). Transfections with this PTM showed a 3 fold increase in restoration of β-gal activity compared to PTM1 or PTM3, respectively (FIG. 3C).

To quantify the trans-spliced mRNA compared to the cis-spliced product, semi-quantitative real time PCR was performed. As expected, control reactions did not show any trans-spliced product, co-transfection of LacZ-T1 and PTM1 yielded 1.9% of repaired lacZ mRNA compared to cis-spliced target. Co-transfection of LacZ-T1 and PTM3 improved trans-splicing to 2.1%. The extension of the binding domain length contained in PTM5 further increased the amount of repaired mRNA to 6.5% of cis-spliced target confirming the results obtained by the β-gal protein assay (Table II).

TABLE II
Relative efficiency of trans-splicing in keratinocytes
measured by semiquantitative real-time PCR^{a}

| Transfection | Cis-splicing^{b} | Trans-splicing | Percentage |
|---|---|---|---|
| T1 | 3.7 | − | |
| PTM3 | − | − | |
| PTM5 | − | | |
| T1 + PTM1 | 3.9 | 0.075 | 1.9% |
| T1 + PTM3 | 4.1 | 0.088 | 2.2% |
| T1 − PTM5 | 4.9 | 0.32 | 6.5% |

^{a}One representative experiment of 5 similar experiments is shown
^{b}Numbers are in nanogram and depict the calculated amounts of DNA

To compare the specificity of trans-splicing between PTMs 1, 3 and 5, a non-specific target placZ-T4 containing mini-intron 9 of the CFTR gene was used. β-gal activity was not significantly increased over basal levels by transfection with pLacZ-T4 or each one of the PTMs alone. Co-transfection of the non-specific target with PTMs 1, 3 and 5 showed a decrease in β-gal activity correlated with changes in their binding domains. With PTM1 the non-specific trans-splicing was ˜12% of specific trans-splicing between CF-Target and CF-PTM14. The PTM with the longest binding domain represented by PTM5 restored only ˜6% the level of β-gal activity compared to that obtained between specific PTM and target.

To achieve protein restoration in COL17A1 harboring the 4003delTC mutation the complete C-terminus 3′ of the mutation has to be incorporated into a PTM and trans-spliced into the mutant pre-mRNA by the spliceosome. To evaluate if this can be achieved, a COL17A1 mini-gene construct spanning exon 51, intron 51 and exon 52 including the addition of a FLAG-sequence at the 3′ end (T2; FIG. 2B) was transfected into 293T cells. To demonstrate the functionality of the mini-gene target, the cis-spliced mRNA derived from this construct was analyzed by RT-PCR and sequenced showing correct length of 568 bp (FIG. 5A; upper panel). A series of PTMs were constructed based upon the LacZ PTMs described above, incorporating their target binding and trans-splicing domains but replacing the 3′ lacZ exon by the cDNA sequence of COL17A1 exons 52 through 56. These PTMs named PTM2, PTM4 and PTM6 (FIG. 2D) were transfected into 293T cells. No trans-spliced product was detected by RT-PCR reaction using primers Ex51-1F and the exon 53 specific reverse primer KI-53-1R. However, co-transfection of the ColI7A1 mini-gene target (T2) and either PTM 2, 4 or 6 followed by RT-PCR analysis indicated accurate trans-splicing producing the expected 574 bp fragment (FIG. 5A; lower panel). Therefore, trans-splicing produces a RNA spanning from exon 51 to exon 56, replacing exon 52 and the attached FLAG sequence of the mini-gene target pre-mRNA. Sequence analysis showed the accuracy of the trans-splicing between the target pre-mRNA and the PTM. The possibility of DNA-recombination events was analyzed by PCR using primers Ex51-1F and KI-53-1R. No product was obtained eliminating the possibility of DNA-recombination events.

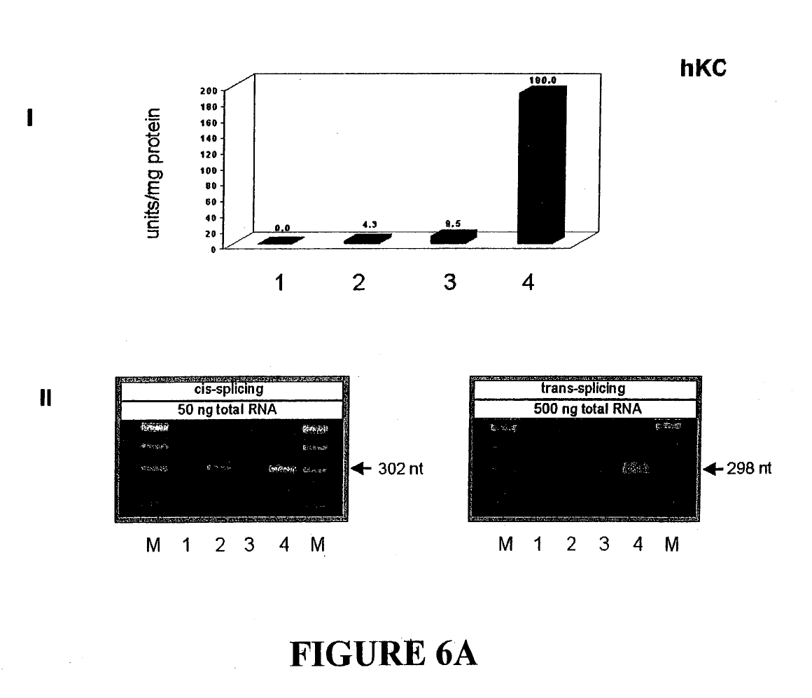

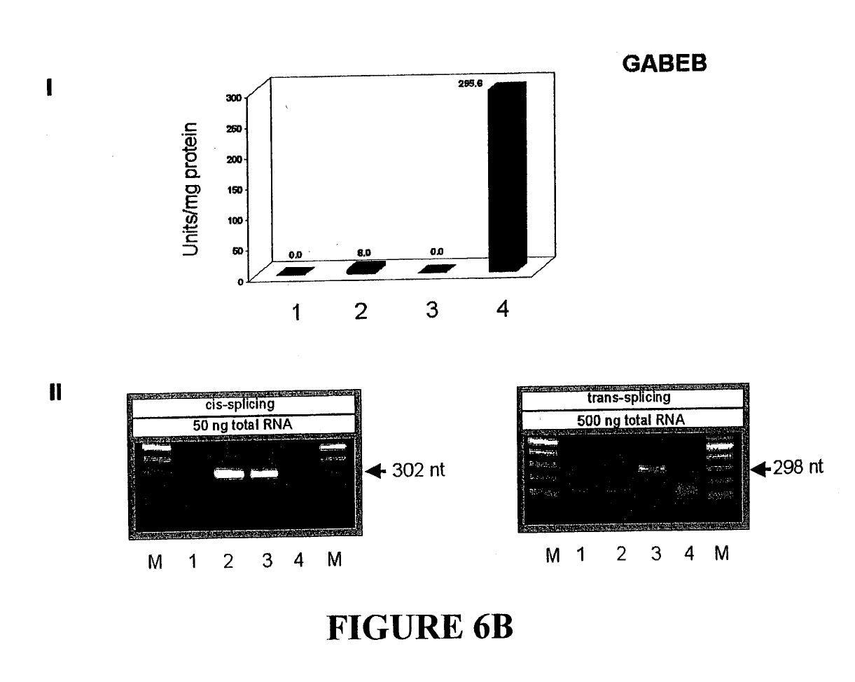

To evaluate if the keratinocyte-specific environment allows for trans-splicing to occur, the LacZ repair system was used in human keratinocytes. First LacZ-T1 or PTM5 alone were transfected into human keratinocytes which did not increase the level of β-gal activity beyond the levels measured in mock transfected keratinocytes. β-gal protein quantification produced a ˜100 fold increase in β-gal activity over background due to mRNA repair by trans-splicing PTM5 pre-mRNA into LacZ-T1 pre-mRNA (FIG. 6A; I). Cis-splicing of the target was detected by RT-PCR analysis of total RNA using primers Lac9F and KI-3R (FIG. 6A; II, left panel). Primer pair Lac9F and KI-5R were utilized for analysis of trans-splicing (FIG. 6A; II, right panel).

Trans-splicing in an immortalized Col17A1 deficient KC cell-line. Transfection of either LacZ-T1 or PTM5 alone produced no β-gal activity, nor positively stained cells in cell culture. Co-transfection of LacZ-T1 and PTM5 produced significant levels of β-gal activity (295 U/mg protein) (FIG. 6B; I). In this cell type cis- and trans-splicing was detected using primer Lac9F and KI-3R (cis) or Lac9F and KI-5R (trans) (FIG. 6B; II). In addition β-gal positive cells could be detected when LacZ-T1 and PTM5 were co-transfected in the immortalized GABEB cell line (FIG. 4, lowest panel).

In both primary keratinocytes and the GABEB cell-line DNA, recombination events were ruled out by PCR analysis as described above. Additional sequence analysis for both cell types showed accurate trans-splicing, with replacement of the stop codon containing exon.

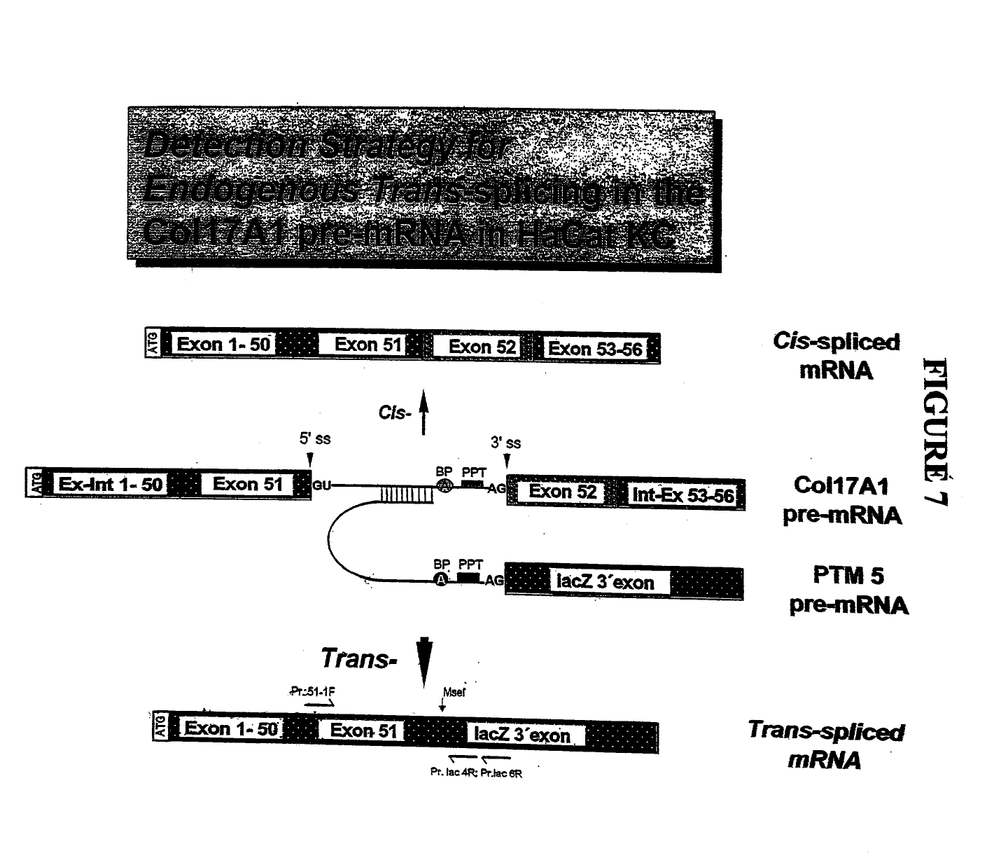

The detection strategy for endogenous trans-splicing of the Col17A1 pre-mRNA in HaCatKC cells is shown in FIG. 7. The pre-trans-splicing molecule (PTM5) which consists of a Col7A1 binding domain 51, spacer element, branch point (BP) and polypyrimidine tract (PPT) followed by a functional part of β-galactosidase lacZ 3′ exon cloned into pcDNA3.1(−) is depicted. This construct was transfected into HaCat cells. Pre-mRNA resulted in correct endogenously trans-spliced product of a genomic fragment spanning exon 1-51 and LacZ 3′ exon confirmed by semi-nested RT-PCR with primer 51-1F, lac6R and lac4R. FIG. 8A depicts the sequence of correct endogenously trans-spliced splice junction of genomic fragment exon 51 with lacZ 3′ exon and confirmation with restriction enzyme digest of 226 bp RT-PCR product with Msel resulting in two fragments of 168 bp and 58 bp. (B).

### 7. EXAMPLE

### Trans-Splicing of the Plectin Target Pre-mRNA

The subsection below describes experiments designed to mediate a trans-splicing reaction between a PTM and a plectin pre-mRNA molecule using 5′ trans-splicing.

### 7.1 Materials and Methods

Isolation and in-vitro culture of keratinocytes. Human keratinocytes are isolated from skin samples after skin biopsy, incubated with dispase at 4° C. overnight and then trypsinized to obtain a single cell suspension. Cells cultured in KGM keratinocyte medium (BioWhitacker, Vervier, Belgium) at 0,15 mM Ca2+.

Cells and cell-lines. Patient keratinocytes from EBS-MD patients are collected by biopsies under local anesthesia, prepared using trypsin, expanded and frozen at different passage numbers in liquid nitrogen. Those keratinocytes with the plectin genetic background are immortalized using HPV16 E6 and HPV7 under the control of an actin promoter (provided by H. Lochmuller, Institute of Biochemistry-Genecenter, LMU, Munich, Germany).

Organotypic culture. Cadaver skin is obtained from a skin bank. The skin is tested to determine that the skin is HIV- and Hepatitis-B negative. Cryopreserved skin is subjected to rapid freeze-thaw cycles in liquid nitrogen to devitalize the cells, washed in sterile PBS and incubated at 37° C. in sterile PBS with antibiotics. Epidermis is removed. The acellular dermis is cut into pieces and each piece is placed into a tissue culture dish papillary side up. Transiently PTM-transfected plectin-deficient keratinocytes are placed on the dermis and grown submerged for one week which yielded a three- to five-cell layer. The skin composite is then lifted to the air-liquid surface, and grown for various periods of times and then analyzed.

Northern blotting. Cultured cells are trypsinized, lysed and RNA is isolated using anion-exchange columns (Qiagen, Hilden, Germany). Isolated RNA is electrophoresed and transferred to a nylon-membrane. The membrane is probed with ³²P-labeled cDNA fragments. The blots are washed and specific bands are detected by exposure to X-ray films. Probes are made using primers designed according to published sequences. After RT-PCR the fragments are subcloned into a pUC18 plasmid and amplified according to standard procedures.

Immunofluorescence. Cultured keratinocytes are fixed with 3% paraformaldehyde. A plectin specific first step antibody (5B3, kindly provided by G. Wiche, Vienna, Austria) and a FITC-labeled secondary antibody is then applied to the sample. After a washing step the respective anti mouse-FITC labeled and anti-rat-Rhodamine labeled second step antibodies are applied. Slides are mounted and immunofluorescence is detected using a Zeiss microscope. The epidermis from organotypic culture will be snap-frozen and cut with a cryostat.

Western Blot analysis. Confluent cells are washed with PBS and scraped off the plate. Cell pellets are lysed in 50 mM Tris-HCl pH 7.5, 150 mM NaCl, 1% (v/v) Triton-X 100, 0.1% (w/v) SDS, 0.5 mM EDTA, 10 μM leupeptin, 100 μM phenylmethylsulfonylfluoride, 100 μM DTT. The epidermis from organotypic culture is lysed directly. 20 μg protein of control and test KC are loaded on a 5% SDS Polyacrylamide Gel. Following electrophoresis, proteins are transferred to nitrocellulose (Hybond C pure; Amersham Pharmacia Biotech, Little Chalfont, UK) in 48 mM Tris-HCl, 39 mM Glycine, 20% (v/v) MeOH, 0.037% (w/v) SDS. The primary monoclonal antibody 5B3-is diluted 1:3 in blocking buffer (200 mM Tris-HCl pH 7.6, 137 mM NaCl, 0.2% (w/v) I-Block, 0.1% (v/v) Tween 20). Immunodetection is monitored with the Western-Star™ Chemiluminescent Detection System (Tropix Inc., Bedford, Mass., USA) following the manufacturer's instructions.

| | |
|---|---|
| Primers: | |
| PLEC-FN: | 5′ GGG AGC TGG TGC TGC TGC TGC TTC 3′ |
| PLEC-FM: | 5′ GGG AGC TGG TGC TGC TGC TGC TGC 3′ |
| PLEC-R: | 5′ CTC TCA AAC TCG CTG CGG AGC TGC 3′ |

Cloning of the Exon 9 to Exon 10 region from plectin gene. The DNA sequence of the plectin gene spanning exon 9 to exon 10 is amplified by using exon 9 upstream and exon 10 downstream primers. For a directional cloning of the fragment restriction sites are added to the primers. The amplified DNA fragment was cloned into a pGEM-3Zf(+) vector (Promega, Madison, USA). The region of exon 9/intron 9/exon 10 is sequenced to affirm the correct sequence. Also the genomic exon 9 to 10 region from the EBS-MD patient is cloned according to this protocol.

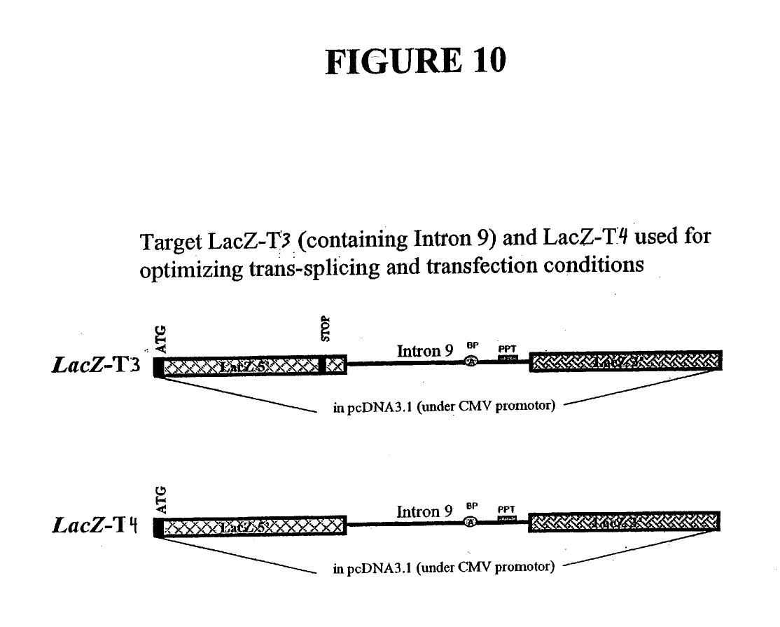

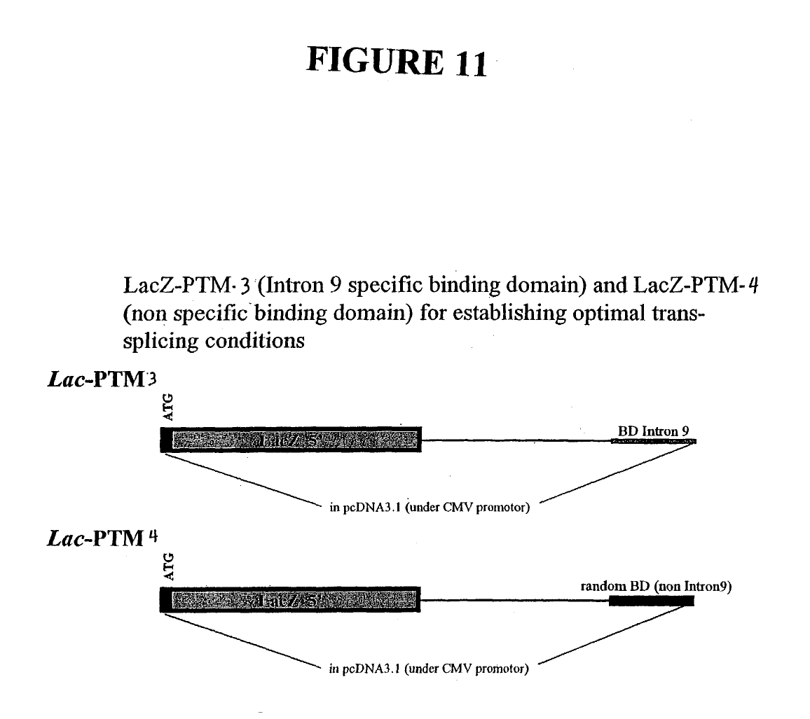

Construction of LacZ vectors and PTM's for trans-splicing mediated gene repair. To select for the best binding requirements in intron 9 of the plectin gene artificial chimeric LacZ targets are constructed (LacZ-T3+T4; FIG. 10) consisting of: 5′ fragment (5′-exon 1-1788 bp) of the LacZ coding sequence with an insertion of two in-frame stop codons at the 3′ end (1761-1762), intron 9 of the plectin gene (PLEC1), and the 3′-exon of-LacZ (1789-3170 bp) (=LacZ-T3); LacZ-T4: 5′ fragment (5′ exon 1-1788 bp) of the LacZ coding sequence, intron 9 of plectin gene (PLEC1), and the 3′exon of LacZ (1789-3170 bp). In addition, repair molecules are constructed, which are referred to as pre-trans-splicing molecules, (Lac-PTM3+4; FIG. 11) LacZ-PTM-3: Binding domain complimentary to intron 9 of PLEC1, a spacer and very strong 5′ splice site elements, followed by the 5′ fragment of LacZ (1-1788 bp) (=PTM-3). PTM-4: Random non intron 9 binding domain followed by the 5′ fragment of LacZ (1-1788 bp).

Construction of the PTM's is performed according to Puttaraju et al., (Mol. Therapy, 2001, 4:105-114) using 5′ splice site elements, a spacer region and a binding domain (BD) complementary to the intron 9 sequence at the 5′ end of the intron to block cis-splicing. Exons 1 through 9 are amplified from cDNA using an exon 1 forward- and an exon 9 reverse-primer. The 5′ PTM domain is attached to the exon fragment using restriction enzymes and ligation PCR technique. For in-vitro studies the PTM's are cloned into a vector containing SP6/T7 promoters (pGEM, pBS) for in vitro RNA synthesis. Furthermore, the PTM's are cloned into a mammalian expression vector (pcDNA 3.1, pcDNA 3.1/His/lacZ,) for in vivo transfection studies in human keratinocytes.

In vitro preparation of RNA. RNA is transcribed using the T7 and/or SP6 promoters on the pGEM-3Zf (+). For the synthesis, a T7/SP6 RNA synthesis kit (Promega) is used. 0.5 to 1 μg of template RNA is added to the transcription buffer and a nucleotide mixture (10 mM each). After 60 min. at 30° C. RNase free DNase I is added to remove template DNA to avoid later interference of template DNA. The reaction is followed by gel purification using 4-8% PAGE to obtain RNA of homogeneous size. After overnight elution the RNA is precipitated.

In vitro splicing and trans-splicing using HeLa extracts. In vitro synthesized and gel purified PTM-RNA and target pre-mRNA is annealed after denaturing at 95-98° C. followed by a slow cooling to 30-34° C. 4 μl of annealed RNA complex, 1× splice buffer and 4 μl of HeLa nuclear extract (Promega) in a final volume of 12.5 μl is incubated at 30° C. for the time indicated. The reaction is stopped by adding an equal volume of high salt buffer. Nucleic acids is purified by phenol:chloroform extraction followed by ethanol precipitation. β-globin pre-mRNA was used as a positive control.

Reverse transcription (RT) PCR. RT-PCR is performed using rTth (if it is from Perkin Elmer) polymerase. Each reaction contains approximately 10 ng of the spliced RNA or 1-2 μg of total RNA. Enzyme buffer, 2.5 mM dNTP's, 10 pM 3′ and 5′ specific primer and 5U of enzyme are added to a reaction volume of 30 μl. RT-reaction is performed at 60° C. for 45 min. Resulting cDNA is amplified by PCR using specific a specific exon primer.

Sequencing of RT-PCR products. The trans-spliced RT-PCR products are reamplified using a specific nested primer and the Perkin Elmer sequencing kit for cycle sequencing using dye-termination mix, 3-10 pmol/μl primer and 360 ng-1.5 μg DNA. After cycle sequencing the reaction is precipitated with ethanol to remove unincorporated nucleotides and to reduce salt concentration. The pellet is dissolved in 25 μl TSR (Template suppression reagent) followed by a 2-3 min. denaturation at 95° C. The sequencing reactions are analyzed using an ABI Prism 310 Sequencer (Perkin Elmer, Foster City, Calif.).

Transfection and cotransfection of target and PTM's into keratinocytes. Keratinocytes are grown as described above. Cells are transfected with PTM 's and target constructs for measuring cis- and trans-splicing efficiencies using lipofectamine following the manufacturer's protocol. Since transfection efficiency is crucial to these experiments a number of different liposomal transfection reagents have been evaluated. Fugene 6 (Roche Diagnostics, Mannheim, Germany) yields significantly improved transfection efficiency in KC (data not shown). In addition, electroporation is employed to further improve on transfection efficiency.

Construction of a cDNA library and 3′RACE.

3′RACE. Because of the known sequence of exon 9 it is possible to clone each exon 9 containing mRNA by 3′RACE (Volloch, V et al., 1994, Nucl Acid Res 22:2507-2511). To generate 3′ end-cDNA clones, reverse transcription (primer extension) is carried out to generate first-strand products. Amplification is achieved using a forward primer specific for exon 9 and an oligo-dT reverse primer to form the second strand of cDNA. Then PCR fragments are cloned and sequenced.

cDNA library. First strand cDNA is synthesized using an oligo-dT primer and M-MLV reverse transcriptase. 2-5 μg of polyadenylated RNA is heated for 65° C. for 5 min and chilled on ice. RT-buffer, 8 mM dNTPs, 2 μg oligo-dT primer, 25μ RNasin and 200μ M-MLV RT is added and incubated for 1 h at 37° C. followed by a RNase H digestion. Excess primer is removed using spin filters. An aliquot of the cDNA is amplified using a nested exon 9 specific primer and oligo dT primer. Obtained products are flushed using Klenow enzyme or T4 DNA polymerase and cloned for sequence analysis.

### 7.2 Results

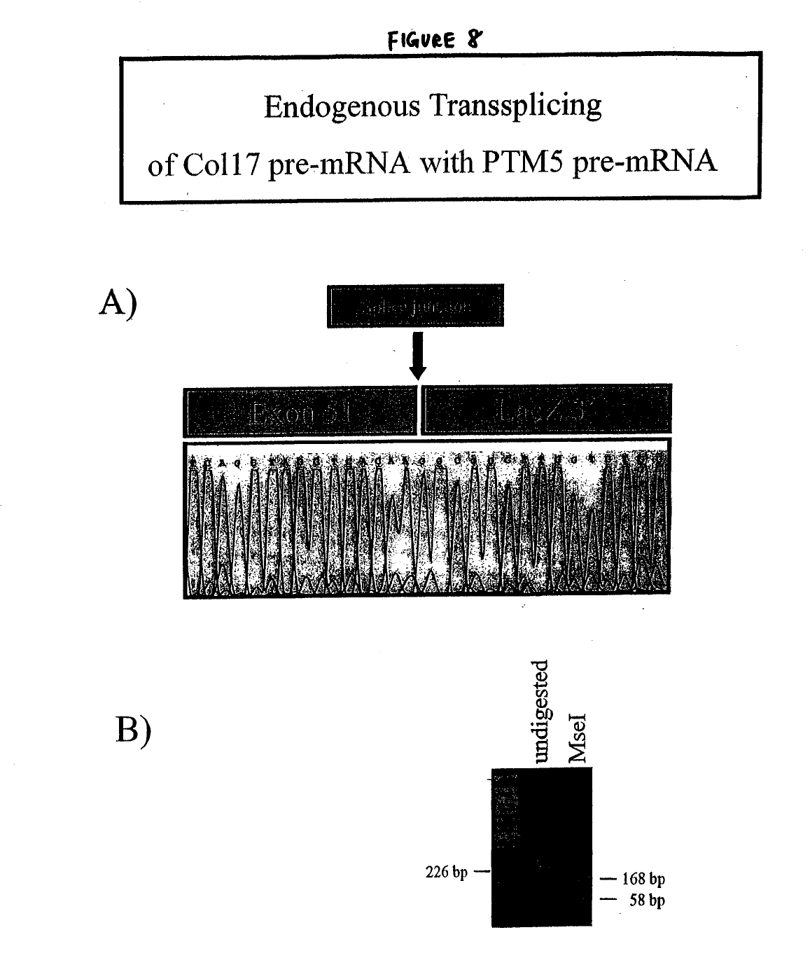

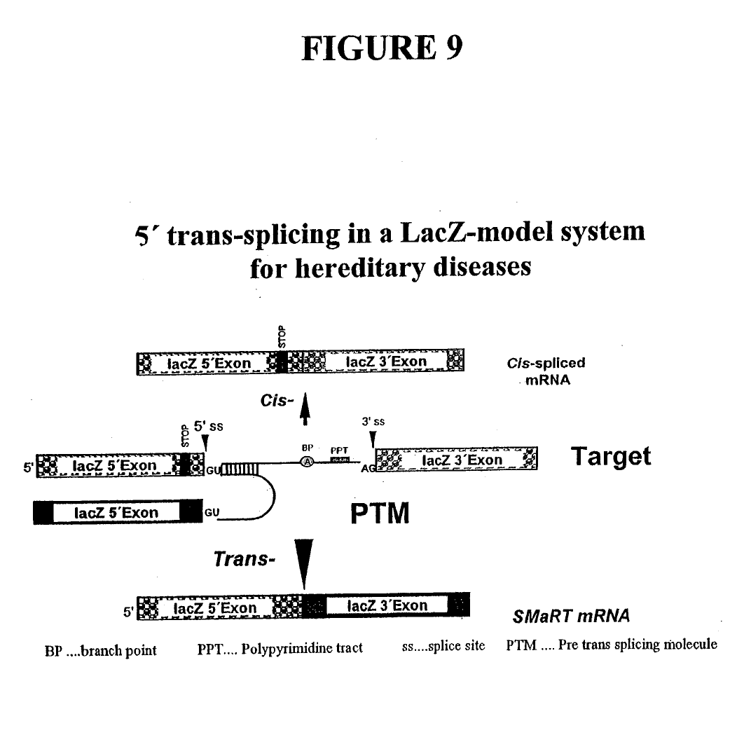

Trans-splicing in a LacZ system. For gene correction of the plectin 1287ins3 mutation, a 5′lacZ model system is used. The corrected fragment for 5′ trans-splicing is only 1356 bp long as opposed to 12833 bp for 3′ trans-splicing (FIG. 9).

Accurate trans-splicing between LacZ-T3 and LacZ-PTM3 leads to the production of a functional mRNA that produces into significant levels of β-galactosidase activity in the LacZ system since the stop codon introduced in the 5′LacZ fragment is eliminated. β-galactosidase activity is not expected when PTM or target constructs are transfected alone.

Based on the results obtained when the LacZ-T3 and LacZ PTM3 are co-transfected, appropriate controls for transfection and splicing efficiency using a construct with plectin intron 9 inserted into the LacZ reading frame is transfected (LacZ-T4) into cells (FIG. 10). This transfection will yield a functional β-galactosidase without co-transfection upon cis-splicing. Furthermore, comparison of targeted vs. non-targeted (non-targeted PTM contains random sequence in place of plectin binding domain; LacZ-PTM4; FIG. 11) trans-splicing will indicate the specificity at the RNA level (RT-PCR analysis) as well as at the protein level (β-galactosidase activity). Variation in the length of the binding domain, inclusion of nonspecific sequences and other modifications in the trans-splicing domain binding sequences will provide important information on the most efficient PTM sequences.

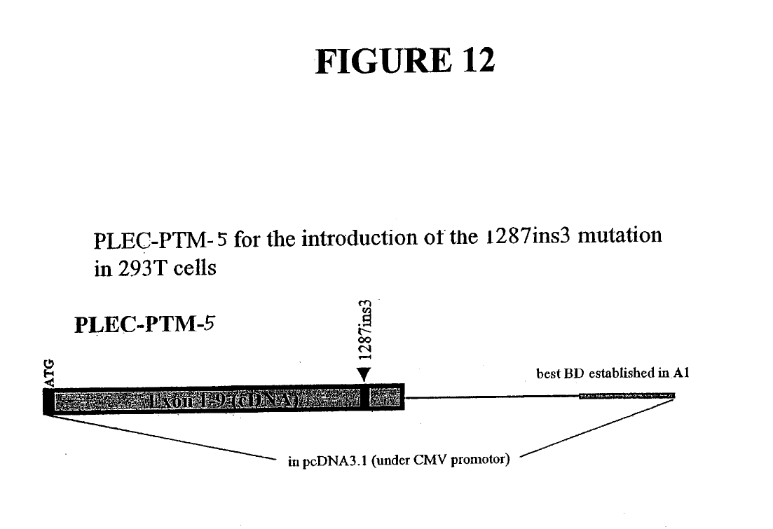

Trans-splicing in cell culture. The efficiency of PTM-induced trans-splicing versus cis-splicing is evaluated in a nonselected transient transfection assay. 293T cells are transfected with a mammalian expression vector containing a plectin PTM-5 (FIG. 12) containing the binding domain found to be spliced most efficiently and harboring exons 1-9 including the 1287ins3 mutation (FIG. 12, PLEC-PTM-5). Total RNA is isolated 48 h post transfection and analyzed by RT-PCR using primers. The amplified product is sequenced, to confirm that PTM-driven trans-splicing occurs in these cells at the predicted splice sites. Cis-splicing is detected by primers PLEC-R and PLEC-FN. Trans-splicing is detected by primer pair PLEC-R and PLEC-FM. Trans-splicing should be detected in a 50 ng total RNA sample. The cis-spliced products can be discriminated in the same RNA pool from trans-spliced products by a 3 bp length difference. No trans-splicing is expected in cells transfected with either target alone or control plasmids alone. The efficiency of PTM-mediated RNA trans-splicing versus cis-splicing is evaluated by a semi-quantitative RT-PCR with increasing amounts of total-RNA using cis- and trans-specific primers (see above). To exclude the possibility of recombination between the target and PTM-plasmids, total DNA was isolated from 293T cells transfected with PLEC-PTM-5 plasmids. PCR is performed with the same primers (PLEC-R and PLEC-FM) used for reverse transcription PCR to detect trans-splicing between the endogenous plectin gene and PLEC-PTM-5.

Evaluation of nonspecific trans-splicing by 3′ RACE and cDNA-library construction in 293T cells. To determine the specificity of PTM's, i.e., whether they are trans-spliced into other endogenous RNAs, 3′ RACE is used to amplify the sequence of all trans-spliced reaction sites. Specifically, reverse transcription will be initiated from an oligo-dT primer. Resulting cDNAs are amplified using a nested exon 9 primer and an oligo dT primer. The amplified products are cloned and sequenced. In addition, cDNA libraries can be constructed from transfected cells to detect illegitimate trans-splicing using a standard dT approach (Sambrook, J et al., 1989 Molecular Cloning: A Laboratory Manual, Cold Spring Harbor Laboratory, Cold Spring Harbor, N.Y.). Individual clones will be checked for sequences specific to the PLEC-PTM-3 construct.

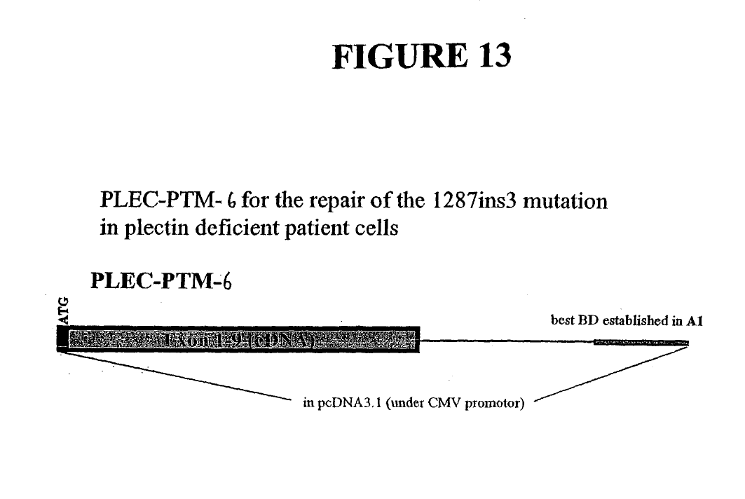

Trans-splicing in Keratinocyte cell-culture. Gene-correction in keratinocytes from plectin-deficient patients by transient transfection. Spliceosome mediated RNA trans-splicing PTMs are designed that are capable of repairing mutated plectin pre-mRNA in patient cells. Since the amount of endogenous plectin mRNA is not reduced in these patient cells (Bauer et al., 2001, Am J. Pathol 158:617-625) there should be no reduction of plectin pre-mRNA containing the required intron 9 pre-mRNA sequences. Based upon information obtained from preliminary experiments, new PTMs are constructed that contain sequences encoding the complete 5′ end of plectin from exons 1 through 9 (PLEC-PTM-6). These constructs are tested by RT-PCR for RNA repair in plectin deficient cells (FIG. 13). Efficiency of trans-splicing versus cis-splicing are assayed using cis- and trans-specific primers. RT-PCR products are sequenced to verify proper splicing between PLEC-PTM-6 and target. PCR of the total cellular DNA (with no Reverse Transcription step) is analyzed to rule out homologous recombination. The specificity of each PTM in trans-splicing to target versus non-target is examined by performing 3′ RACE followed by the cloning and sequencing of a number of clones. PTM specificity is examined for PLEC- PTM-6 and its derivatives.

Inclusion of a safety domain into the binding domain is known to decrease nonspecific trans-splicing, thus, a second type of plectin PTM is also developed, the plectin-safety-PTM. The binding domain of this safety PTM has complementarity to regions of the PTM's splice site (PPT and BP), and has insertions to form a stem structure, which is designed to block access of splicing factors to the PTM splice site. A portion of the PTM binding domain left as single-stranded initiates contact with a target pre-mRNA. Upon binding to the target through base-pairing, the safety is predicted to unwind exposing the splicing elements which are now ready for binding with splicing factors.

Gene-repair and restoration of protein function in 1287ins3-plectin-deficient keratinocytes from patients on protein level. After transfection of the improved PTM into patients' keratinocytes, expression of plectin is evaluated by immunofluorescence analysis and Western blotting.

Trans-splicing in an organotypic culture. A composite skin is cultured as described above. The expression vector containing the improved PTM as determined above is used in these experiments. The growing composite skin equivalent is analyzed at time points day 1-5 after being lifted to the air by immunfluorescence for correct expression of plectin.

The present invention is not to be limited in scope by the specific embodiments described herein. Indeed, various modifications of the invention in addition to those described herein will become apparent to those skilled in the art from the foregoing description and accompanying Figures. Such modifications are intended to fall within the scope of the appended claims. Various references are cited herein, the disclosure of which are incorporated by reference in their entireties.

We claim:

1. A cell of the skin comprising a nucleic acid molecule wherein said nucleic acid molecule comprises:
a) one or more target binding domains that target binding of the nucleic acid molecule to a pre-mRNA expressed within the cell of the skin;
b) a 3′ splice region comprising a branch point and a 3′ splice acceptor site;
c) a spacer region that separates the 3′ splice region from the target binding domain; and
d) a nucleotide sequence to be trans-spliced to the target pre-mRNA;
wherein said nucleic acid molecule is recognized by nuclear splicing components within the cell of the skin.

2. A cell of the skin comprising a nucleic acid molecule wherein said nucleic acid molecule comprises:
a) one or more target binding domains that target binding of the nucleic acid molecule to a pre-mRNA expressed within the cell of the skin;
b) a 3′ splice acceptor site;
c) a spacer region that separates the 3′ splice region from the target binding domain; and
d) a nucleotide sequence to be trans-spliced to the target pre-mRNA;
wherein said nucleic acid molecule is recognized by nuclear splicing components within the cell of the skin.

3. A cell of the skin comprising a nucleic acid molecule wherein said nucleic acid molecule comprises:
a) one or more target binding domains that target binding of the nucleic acid molecule to a pre-mRNA expressed within the cell of the skin;
b) a 5′ splice site;
c) a spacer region that separates the 5′ splice site from the target binding domain; and
d) a nucleotide sequence to be trans-spliced to the target pre-mRNA;
wherein said nucleic acid molecule is recognized by nuclear splicing components within the cell of the skin.

4. The cell of claim 1 wherein the nucleic acid molecule further comprises a 5′ donor site.

5. The cell of claim 1 wherein the 3′ splice region further comprises a pyrimidine tract.

6. The cell of claim 1, 2 or 3 wherein said nucleic acid molecule further comprises a safety sequence comprising one or more complementary sequences that bind to one or both sides of the 5′ splice site.

7. The cell of claim 1, 2 or 3 wherein the nucleic acid molecule further comprises a safety nucleotide sequence comprising one or more complementary sequences that bind to one or more sides of the 3′ splice region.

8. The cell of claim 1 wherein the binding of the nucleic acid molecule to the target pre-mRNA is mediated by complementary, triple helix formation, or protein-nucleic acid interaction.

9. The cell of claim 1 wherein the nucleotide sequences to be trans-spliced to the target pre mRNA encodes a polypeptide expressed within the cell of the skin.

10. The cell of claim 9 wherein the polypeptide is a keratinocyte specific polypeptide.

11. The cell of claim 9 wherein the polypeptide is a melanocyte specific polypeptide.

12. The cell of claim 9 wherein the polypeptide is selected from the group consisting of a plectin, type VII collagen, type XVII collagen, and laminin polypeptide.

13. The cell of claim 1 wherein said cell is a cancer cell of the skin.

14. The cell of claim 10 wherein said cell is a melanoma or basal cell carcinoma cell.

15. The cell of claim 1 wherein said cell is selected from the group consisting of a keratinocyte, melanocyte and dermal papilla cell.

16. A cell of the skin comprising a recombinant vector wherein said vector expresses a nucleic acid molecule comprising:
a) one or more target binding domains that target binding of the nucleic acid molecule to a pre-mRNA expressed within the cell of the skin;
b) a 3′ splice region comprising a branch point and a 3′ splice acceptor site;
c) a spacer region that separates the 3′ splice region from the target binding domain; and
d) a nucleotide sequence to be trans-spliced to the target pre-mRNA;
wherein said nucleic acid molecule is recognized by nuclear splicing components within the cell of the skin.

17. A cell of the skin comprising a recombinant vector wherein said vector expresses a nucleic acid molecule comprising:
a) one or more target binding domains that target binding of the nucleic acid molecule to a pre-mRNA expressed within the cell of the skin;
b) a 3′ splice acceptor site;
c) a spacer region that separates the 3′ splice region from the target binding domain; and
d) a nucleotide sequence to be trans-spliced to the target pre-mRNA;
wherein said nucleic acid molecule is recognized by nuclear splicing components within the cell of the skin.

18. A cell of the skin comprising a recombinant vector wherein said vector expresses a nucleic acid molecule comprising:
a) one or more target binding domains that target binding of the nucleic acid molecule to a pre-mRNA expressed within the cell of the skin;
b) a 5′ splice site;
c) a spacer region that separates the 5′ splice site from the target binding domain; and
d) a nucleotide sequence to be trans-spliced to the target pre-mRNA;
wherein said nucleic acid molecule is recognized by nuclear splicing components within the cell of the skin.

19. The cell of claim 16 wherein the nucleic acid molecule further comprises a 5′ donor site.

20. The cell of claim 16 wherein the 3′ splice region further comprises a pyrimidine tract.

21. The cell of claim 16, 17 or 18 wherein the nucleic acid molecule further comprises a safety nucleotide sequence comprising one or more complementary sequences that bind to one or more sides of the 3′ splice region.

22. A method of producing a chimeric RNA molecule in a cell of the skin comprising:
contacting a target pre-mRNA expressed in the cell of the skin with a nucleic acid molecule recognized by nuclear splicing components wherein said nucleic acid molecule comprises:
a) one or more target binding domains that target binding of the nucleic acid molecule to a pre-mRNA expressed within the cell of the skin;
b) a 3′ splice region comprising a branch point and a 3′ splice acceptor site;
c) a spacer region that separates the 3′ splice region from the target binding domain; and
d) a nucleotide sequence to be trans-spliced to the target pre-mRNA;
under conditions in which a portion of the nucleic acid molecule is trans-spliced to a portion of the target pre-mRNA to form a chimeric RNA within the cell of the skin.

23. A method of producing a chimeric RNA molecule in a cell of the skin comprising:
contacting a target pre-mRNA expressed in the cell of the skin with a nucleic acid molecule recognized by nuclear splicing components wherein said nucleic acid molecule comprises:
a) one or more target binding domains that target binding of the nucleic acid molecule to a pre-mRNA expressed within the cell of the skin;
b) a 3′ splice acceptor site;
c) a spacer region that separates the 3′ splice region from the target binding domain; and
d) a nucleotide sequence to be trans-spliced to the target pre-mRNA; under conditions in which a portion of the nucleic acid molecule is trans-spliced to a portion of the target pre-mRNA to form a chimeric RNA within the cell of the skin.

24. A method of producing a chimeric RNA molecule in a cell of the skin comprising:
contacting a target pre-mRNA expressed within the cell with a nucleic acid molecule recognized by nuclear splicing components wherein said nucleic acid molecule comprises:
a) one or more target binding domains that target binding of the nucleic acid molecule to a pre-mRNA expressed within the cell of the skin;
b) a 5′ splice site;
c) a spacer region that separates the 5′ splice site from the target binding domain; and
d) a nucleotide sequence to be trans-spliced to the target pre-mRNA;
wherein said nucleic acid molecule is recognized by nuclear splicing components within the cell.

25. The method of claim 22 wherein the nucleic acid molecule further comprises a 5′ donor site.

26. The method of claim 22 wherein the 3′ splice region further comprises a pyrimidine tract.

27. The method of claim 22, 23, or 24 wherein the nucleic acid molecule further comprises a safety nucleotide sequence comprising one or more complementary sequences that bind to one or more sides of the 3′ splice region.

28. The method of claim 22 wherein the nucleotide sequences to be trans-spliced to the target pre-mRNA encodes a polypeptide expressed within the cell of the skin.

29. The method of claim 28 wherein the polypeptide is a keratinocyte specific polypeptide.

30. The method of claim 28 wherein the polypeptide is a melanocyte specific polypeptide.

31. The method of claim 28 wherein the polypeptide expressed within the cell of the skin is selected from the group consisting of a plectin, type VII collagen, type XVII collagen, and laminin polypeptide.

32. The method of claim 22 wherein said cell of the skin is a cancer cell.

33. The method of claim 32 wherein said cell is a melanoma or basal cell carcinoma cell.

34. The method of claim 32 wherein the nucleotide sequence to be trans-spliced to the target pre-mRNA encodes a polypeptide toxic to said cell.

35. The method of claim 22 wherein said cell is selected from the group consisting of a keratinocyte, melanocyte and dermal papilla cell.

36. A nucleic acid molecule comprising:
a) one or more target binding domains that target binding of the nucleic acid molecule to a pre-mRNA expressed within a cell of the skin;
b) a 3′ splice region comprising a branch point and a 3′ splice acceptor site;
c) a spacer region that separates the 3′ splice region from the target binding domain; and
d) a nucleotide sequence to be trans-spliced to the target pre-mRNA;
wherein said nucleic acid molecule is recognized by nuclear splicing components within the cell.

37. A nucleic acid molecule comprising:
a) one or more target binding domains that target binding of the nucleic acid molecule to a pre-mRNA expressed within a cell of the skin;
b) a 3′ splice acceptor site;
c) a spacer region that separates the 3′ splice region from the target binding domain; and
d) a nucleotide sequence to be trans-spliced to the target pre-mRNA;
wherein said nucleic acid molecule is recognized by nuclear splicing components within the cell.

38. A nucleic acid molecule comprising:
a) one or more target binding domains that target binding of the nucleic acid molecule to a pre-mRNA expressed within a cell of the skin;
b) a 5′ splice site;
c) a spacer region that separates the 5′ splice site from the target binding domain; and
d) a nucleotide sequence to be trans-spliced to the target pre-mRNA;
wherein said nucleic acid molecule is recognized by nuclear splicing components within the cell.

39. The nucleic acid molecule of claim 36 wherein the nucleic acid molecule further comprises a 5′ donor site.

40. The nucleic acid molecule of claim 36 wherein the 3′ splice region further comprises a pyrimidine tract.

41. The nucleic acid molecule of claim 36, 37 or 38 wherein the nucleic acid molecule further comprises a safety nucleotide sequence comprising one or more complementary sequences that bind to one or more sides of the 3′ splice region.

42. The nucleic acid molecule of claim 36 wherein the binding of the nucleic acid molecule to the target pre-mRNA is mediated by complementary, triple helix formation, or protein-nucleic acid interaction.

43. The nucleic acid molecule of claim 36 wherein the nucleotide sequences to be trans-spliced to the target pre mRNA encodes a polypeptide expressed within a cell of the skin.

44. The nucleic acid molecule of claim 43 wherein the polypeptide is a keratinocyte specific polypeptide.

45. The nucleic acid molecule of claim 40 wherein the polypeptide is a melanocyte specific polypeptide.

46. The nucleic acid molecule of claim 43 wherein the polypeptide expressed within the cell of the skin is selected from the group consisting of a plectin, type VII collagen, type XVII collagen, and laminin polypeptide.

47. The nucleic acid molecule of claim 36 wherein the nucleotide sequence to be trans-spliced to the target pre-mRNA encodes a polypeptide toxic to said cell.

48. A eukaryotic expression vector wherein said vector expresses a nucleic acid molecule comprising:
a) one or more target binding domains that target binding of the nucleic acid molecule to a pre-mRNA expressed within a cell of the skin;
b) a 3′ splice region comprising a branch point and a 3′ splice acceptor site;
c) a spacer region that separates the 3′ splice region from the target binding domain; and
d) a nucleotide sequence to be trans-spliced to the target pre-mRNA; wherein said nucleic acid molecule is recognized by nuclear splicing components within the cell of the skin.

49. A eukaryotic expression vector wherein said vector expresses a nucleic acid molecule comprising:
a) one or more target binding domains that target binding of the nucleic acid molecule to a pre-mRNA expressed within a cell of the skin;
b) a 3′ splice acceptor site;
c) a spacer region that separates the 3′ splice region from the target binding domain; and
d) a nucleotide sequence to be trans-spliced to the target pre-mRNA;
wherein said nucleic acid molecule is recognized by nuclear splicing components within the cell.

50. A eukaryotic expression vector wherein said vector expresses a nucleic acid molecule comprising:
a) one or more target binding domains that target binding of the nucleic acid molecule to a pre-mRNA expressed within a cell of the skin;
b) a 5′ splice site;
c) a spacer region that separates the 5′ splice site from the target binding domain; and
d) a nucleotide sequence to be trans-spliced to the target pre-mRNA;
wherein said nucleic acid molecule is recognized by nuclear splicing components within the cell.

51. The vector of claim 48 wherein the nucleic acid molecule further comprises a 5′ donor site.

52. The vector of claim 48 wherein the nucleic acid molecule further comprises a pyrimidine tract.

53. The vector of claim 48, 49 or 50 wherein the nucleic acid molecule further comprises a safety nucleotide sequence comprising one or more complementary sequences that bind to one or more sides of the 3′ splice region.

54. The vector of claim 48, 49 or 50 wherein said vector is a viral vector.

55. The vector of claim 44, 43 or 44 wherein expression of the nucleic acid molecule is controlled by a skin cell specific promoter.

56. A composition comprising a physiologically acceptable carrier and a nucleic acid molecule according to any of claims 36-47.

57. The composition of claim 56 wherein said composition is applied to the skin.

58. A method for correcting a genetic defect in a subject comprising administering to said subject a nucleic acid molecule comprising:
a) one or more target binding domains that target binding of the nucleic acid molecule to a pre-mRNA expressed within a cell of the skin wherein said pre-mRNA is encoded by a gene containing a genetic defect; and
b) a nucleotide sequence to be trans-spliced to the target pre-mRNA;
wherein said nucleic acid molecule is recognized by nuclear splicing components within the cell.

59. A method for imaging gene expression in a cell of the skin comprising administering to said subject a nucleic acid molecule comprising:
a) one or more target binding domains that target binding of the nucleic acid molecule to a pre-mRNA expressed within a cell of the skin wherein said pre-mRNA is encoded by a gene containing a genetic defect; and
b) a nucleotide sequence to be trans-spliced to the target pre-mRNA wherein said nucleotide sequence encodes a reporter molecule;
wherein said nucleic acid molecule is recognized by nuclear splicing components within the cell.


SEQUENCE LISTING (31 sequences)

SEQ ID NO: 1 - RNA, 8 nt, Artificial Sequence
  5′ splice site consenus sequence
agguragu

SEQ ID NO: 2 - RNA, 7 nt, Artificial Sequence
  unsure 2: A, C, G or U
ynyurac

SEQ ID NO: 3 - DNA, 258 bp, Artificial Sequence
  5′ fragment sequence of mini-intron
gtagttcttttgttcttcactattaagaacttaatttggtgtccatgtctctttttttttctagtttgtagtgctggaaggtatttttggagaaattcttacatgagcattaggagaatgtatgggtgtagtgtcttgtataatagaaattgttccactgataatttactctagttttttatttcctcatattattttcagtggctttttcttccacatctttatattttgcaccacattcaacactgtagcggccgc

SEQ ID NO: 4 - DNA, 269 bp, Artificial Sequence
  3 fragment sequence of mini-intron
caactatctgaatcatgtgccccttctctgtgaacctctatcataatacttgtcacactgtattgtaattgtctcttttactttcccttgtatcttttgtgcatagcagagtacctgaaacaggaagtattttaaatattttgaatcaaatgagttaatagaatctttacaaataagaatatacacttctgcttaggatgataattggaggcaagtgaatcctgagcgtgatttgataatgacctaataatgatgggttttatttccag

SEQ ID NO: 5 - RNA, 18 nt, Artificial Sequence
  ISAR consensus sequence
gggcugauuuuuccaugu

SEQ ID NO: 6 - DNA, 23 bp, Artificial Sequence
  Oligonucleotide primer
taatacgactcactatagggaga

SEQ ID NO: 7 - DNA, 23 bp, Artificial Sequence
  Oligonucleotide primer
atttaggtgacactatagaagng

SEQ ID NO: 8 - DNA, 23 bp, Artificial Sequence
  Oligonucleotide primer
aattaaccctcactaaagggaga

SEQ ID NO: 9 - DNA, 29 bp, Artificial Sequence
  Oligonucleotide primer
cgggatccgtaggtgccccgacggtgatg

SEQ ID NO: 10 - DNA, 32 bp, Artificial Sequence
  Oligonucleotide primer
ctagggtaaccagggtgagaagctgcatgagt

SEQ ID NO: 11 - DNA, 31 bp, Artificial Sequence
  Oligonucleotide primer
ctaggctagcctgccggcttgtcattcatcc

SEQ ID NO: 12 - DNA, 57 bp, Artificial Sequence
  Oligonucleotide primer
ctagaagcttttacttgtcatcgtcgtccttgtagtcgctgcatgctctctgacacc

SEQ ID NO: 13 - DNA, 52 bp, Artificial Sequence
  Oligonucleotide primer
ctagggtacctcttcttttttttgatatcctgcaggtcctgatgtgcgcagc

SEQ ID NO: 14 - DNA, 32 bp, Artificial Sequence
  Oligonucleotide primer
ctagaagcttttatggagaccttggacctaag

SEQ ID NO: 15 - DNA, 21 bp, Artificial Sequence
  Oligonucleotide primer
atcaaatctgtcgatccttcc

SEQ ID NO: 16 - DNA, 23 bp, Artificial Sequence
  Oligonucleotide primer
gactgatccacccagtcccatta

SEQ ID NO: 17 - DNA, 23 bp, Artificial Sequence
  Oligonucleotide primer
gactgatccacccagtcccagac

SEQ ID NO: 18 - DNA, 20 bp, Artificial Sequence
  Oligonucleotide primer
catcccaggccctccaggac

SEQ ID NO: 19 - DNA, 21 bp, Artificial Sequence
  Oligonucleotide primer
ttgtcatcgtcgtccttgtag

SEQ ID NO: 20 - DNA, 18 bp, Artificial Sequence
  Oligonucleotide primer
gtaggccatcccttgcag

SEQ ID NO: 21 - DNA, 24 bp, Artificial Sequence
  Oligonucleotide primer
gggagctggtgctgctgctgcttc

SEQ ID NO: 22 - DNA, 24 bp, Artificial Sequence
  Oligonucleotide primer
gggagctggtgctgctgctgctgc

SEQ ID NO: 23 - DNA, 24 bp, Artificial Sequence
  Oligonucleotide primer
ctctcaaactcgctgcggagctgc

SEQ ID NO: 24 - DNA, 7 bp, Artificial Sequence
  PTM branch point
tactaac

SEQ ID NO: 25 - DNA, 21 bp, Artificial Sequence
  PTM polypyrimidine tract
ctcttctttttttttctgcag

SEQ ID NO: 26 - DNA, 32 bp, Artificial Sequence
  Binding domain of pCol17PTM1
ggagttagggagtctctcccagggtgtcaatg

SEQ ID NO: 27 - DNA, 27 bp, Artificial Sequence
  Binding domain of pCol17PTM3
gggggagaagctgctgcatgagggagc

SEQ ID NO: 28 - DNA, 52 bp, Artificial Sequence
  Binding domain of pCol17PTM5
aagctgcctgagtgggagctaagatctcggttgagataaagacttgggagtt

SEQ ID NO: 29 - DNA, 30 bp, Artificial Sequence
  cis-spliced product
gtttacagggcggcttcgtgtaataatggg

SEQ ID NO: 30 - DNA, 24 bp, Artificial Sequence
  trans-spliced product
gtttacagggcgccttcgtctggg

SEQ ID NO: 31 - DNA, 30 bp, Artificial Sequence
  trans-spliced product
tcagctacctcacaaggcggcttcgtctgg